(19) United States
(12) Patent Application Publication
(10) Pub. No.: US 2013/0310767 A1
(43) Pub. Date: Nov. 21, 2013

(54) SEAL TIP CATHETER DEVICES OR METHODS

(71) Applicants: Matthew S. Solar, Indialantic, FL (US); Craig J. Pagan, West Melbourne, FL (US); Glen D. Perry, Melbourne, FL (US)

(72) Inventors: Matthew S. Solar, Indialantic, FL (US); Craig J. Pagan, West Melbourne, FL (US); Glen D. Perry, Melbourne, FL (US)

(73) Assignee: C2C Developement, LLC, Melbourne, FL (US)

(21) Appl. No.: 13/885,586

(22) PCT Filed: Nov. 16, 2011
(86) PCT No.: PCT/US11/61042

§ 371 Date: Aug. 2, 2013

Related U.S. Application Data
(60) Provisional application No. 61/414,341, filed on Nov. 16, 2010.

(51) Int. Cl. A61M 25/00; A61M 39/22

(52) U.S. Cl. CPC A61M 25/0043; A61M 39/22
USPC 604/247; 604/256

(57) ABSTRACT

A first catheter can include a first catheter body providing a longitudinal bore and can have flexible first seal at its distal portion. The first seal can at least partially occlude the bore at the distal portion of the catheter body in a first configuration. A second catheter can be slid into or through the bore of the first catheter body, such as to expand the first seal into a second configuration. Wicking or reflux can be inhibited. Other features, applications, and methods can be included.

### CLAIM OF PRIORITY

This patent application claims the benefit of priority, under 35 U.S.C. Section 119(e), to U.S. Provisional Patent Application Ser. No. 61/414,341 entitled “SEALED TIP CATHETER DESIGN AND METHOD” filed on Nov. 16, 2010, which is hereby incorporated by reference herein in its entirety.

### BACKGROUND

A catheter can be inserted into the body of a human or animal patient or subject, such as to help carry out medical diagnosis or treatment, for example, by transporting fluid to or from an internal target location within the subject. In an example, a fluid substance, such as a drug or other therapeutic agent, can be infused into the subject at an internal target location within the subject's brain.

For example, U.S. Patent Publication No. 2010/0069855 to Christopher D. Ross, entitled CLOG-PREVENTING VALVED CATHETER AND METHOD OF USING THE CATHETER, is directed toward an anti-clogging catheter that includes a tubular cannula defining a fluid passageway and shaped and tipped to removably insert within and be removed from a human body.

For another example, U.S. Pat. No. 7,575,971 to Matthew S. Solar et al., entitled INDEXING CELL DELIVERY CATHETER, is directed toward an insertion device that is capable of distributing a media over a large distribution volume inside a patient while reducing an amount of tissue that is disturbed by the procedure.

For another example, U.S. Pat. No. 6,599,274 to John Kucharczyk et al., entitled CELL DELIVERY CATHETER AND METHOD, is directed toward a cell delivery catheter having one or more apertures that is used to position and deliver cells to an implant site in a patient.

For another example, U.S. Patent Publication No. 2011/0106009 to Matthew S. Solar et al., entitled DEVICE AND METHOD FOR DELIVERING THERAPEUTIC AGENTS TO AN AREA OF THE BODY, is directed toward creating one or more channels, tunnels, or grooves, in tissue using a relatively small diameter minimally invasive stylet and then removing the stylet before infusion.

For another example, U.S. Pat. No. 7,371,225 to Edward H. Oldfield et al., entitled METHOD OF CONVECTION ENHANCED DELIVERY OF THERAPEUTIC AGENTS, is directed toward a method for monitoring and controlling convection-enhanced delivery of a therapeutic agent to a target tissue.

### OVERVIEW

The present inventors have recognized, among other things, a need for improved catheter devices or methods, such as for helping carry out medical diagnosis or treatment, for example, by transporting fluid to or from an internal target location within the subject. In an example, the present devices or methods can be used to help infuse a fluid substance, such as a drug or other therapeutic agent, into the subject to an internal target location within the subject's brain, such as to help inhibit coring or otherwise damaging delicate brain tissue during catheter insertion or removal or during acute or chronic fluid delivery to the target location using the catheter.

For example, the present inventors have recognized, among other things, that when delivering a therapeutic agent to a patient, such as a drug, a cell line or viral vector or other biologic, or other substance, it can be important to control the volume or dose of the substance that is delivered. The present inventors have recognized, among other things, that a common problem when delivering a liquid or other fluid therapeutic agent through a catheter into brain parenchyma can be “reflux,” which can describe the flow of the fluid back up into the space between the catheter and the tissue, or, when both an inner and an outer catheter are used, reflux can additionally or alternatively describe flow of the fluid back up into the space between the inner and outer catheter, which can be substantial, such as due to capillary surface tension “wicking.”

An Example can include subject matter (such as an apparatus, a method, a means for performing acts, or a machine-readable medium including instructions that, when performed by the machine, cause the machine to perform acts) that can include or use a medical apparatus. The medical apparatus can include a first catheter. The first catheter can include a first catheter body that can include a proximal portion, a distal portion, and a longitudinal bore extending between the proximal portion of the first catheter body and the distal portion of the first catheter body, the longitudinal bore defining an inner circumferential periphery. The first catheter can include an elastically deformable first seal, located at the distal portion of the first catheter body. The first seal can be more flexible than a more proximal portion of the first catheter body. The first seal can define an inner circumferential periphery that, in a first configuration, can be laterally smaller than the inner circumferential periphery of a more proximal portion of the bore to at least partially occlude the bore at the distal portion of the catheter body and, in a second configuration, the inner circumferential periphery of the first seal can be expandable relative to the first configuration.

An Example can include a second catheter. The second catheter can include a second catheter body that can include a proximal portion, a distal portion, and a longitudinal bore extending between the proximal portion of the second catheter body and the distal portion of the second catheter body. At least a portion of the second catheter body can be sized and shaped to be inserted into or through and to slidingly engage the bore of the first catheter body. The distal portion of the second catheter can be laterally larger than the inner circumferential periphery of the first seal in its first configuration such as to expand the first seal into its second configuration. An elastically deformable second seal can be located at or toward the proximal portion of at least one of the first or second catheter body. The second seal can be configured to be elastically deformable to conformally encompass an outer periphery of a proximal portion of the other one of the first or second catheter body when at least a portion of the second catheter has been inserted into or through the bore of the first catheter body.

An Example can include a second catheter. The second catheter can comprise a second catheter body including proximal portion, a distal portion, and a longitudinal bore extending between the proximal portion of the second catheter body and the distal portion of the second catheter body. At least a portion of the second catheter body can be sized and shaped to be inserted into or through and to slidingly engage the bore of the first catheter body. The distal portion of the second catheter can be laterally larger than the inner circumferential periphery of the first seal in its first configuration to expand the first seal into its second configuration. A second stylet can have a distal portion that is laterally smaller than the inner circumferential periphery of the bore of the second catheter body. The second stylet can be configured to be capable of being inserted into or through and to slidingly engage the bore of the second catheter body. The distal portion of the second stylet can include a shape-memory property such that the distal portion of the second stylet can curve away from a trajectory of the longitudinal bore of the second catheter body when the distal portion of the second stylet is extended beyond a distal end of the second catheter body. The apparatus can be configured such that the second catheter body can be longitudinally straight when the second stylet is retracted within the bore of the second catheter body.

An Example can include a pressure-relief valve assembly, which can be configured to be in fluid communication with the longitudinal bore of the second catheter. The pressure relief valve assembly can be configured to open in response to a fluid pressure within the longitudinal bore of the second catheter meeting or exceeding a specified maximum pressure threshold value.

An Example can include at least one of the first catheter body or the second catheter body including different first and second materials providing different respective first and second stiffnesses. The second material can be stiffer than the first material. The second material can be segmented into a series of separate or interconnected segments in a longitudinal direction.

Each of these non-limiting examples can stand on its own, or can be combined with one or more of the other examples in any permutation or combination. This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of portions of an instrument that can include an outer first catheter body, an inner second catheter body, an obturator or outer first stylet, and an obturator or inner second stylet, and an example of an insertion arrangement of these components.

FIG. 2 shows a cross-sectional view of an example of the outer first catheter, including the distal portion of the outer first catheter.

FIG. 3 shows an example of the distal portion of the outer first catheter with a rounded or blunt distal end of the first stylet extending slightly beyond the distal end of the first seal.

FIG. 4 shows a cross-sectional view of an example of the distal portion of the outer first catheter.

FIGS. 5A and 5B show an example in which an outer dimension of the outer periphery of the first seal can be expanded from that of a first configuration, such as shown in FIG. 5A.

FIGS. 6A-6B show an example in which the distal edge of the first seal can be actuated from a substantially orthogonal angle in a first configuration, such as shown in FIG. 6A, to an acute angle in a second configuration, such as shown in FIG. 6B.

FIG. 7 shows an example of a proximal elastically deformable second seal that can additionally or alternatively be provided, such as to help inhibit or prevent infusate reflux into the clearance space between (1) the outer periphery of the inner second catheter and (2) the inner periphery of the outer first catheter.

FIG. 8 shows an example of portions of the handle that can include a Y-connector or a T-connector body.

FIGS. 9A and 9B show an example of a compression element of a head portion of the handle, which can be provided by a Tuohy-Borst connector.

FIG. 10 shows an example of a pressure-relief valve assembly.

FIGS. 11A and 11B show an example of a distal portion of an inner second catheter, wherein FIG. 11B shows a cross-sectional view of the distal portion of the inner second catheter that is shown in perspective in FIG. 11A.

FIGS. 12A and 12B show an example of a distal portion of an example of a catheter body, such as can be provided by an inner second catheter.

FIGS. 13A, 13B, 13C, and 13D show an example of a distal portion of a catheter, such as can be provided by the inner second catheter, and in which an opening can be configured to be opened and closed.

FIGS. 14, 15, 16, and 17 show examples of how at least one of the outer first catheter body or the inner second catheter body can be constructed so as to include different first and second materials providing different respective first and second stiffnesses.

FIGS. 18A, 18B, and 18C show an example of a distal portion of a second catheter, such as can be provided by the inner second catheter.

FIG. 19A shows an example of a distal portion of a second catheter, such as can be provided by the inner second catheter, and which can include an opening that can be covered by a slitted membrane or other covering that can include one or more slits.

FIGS. 19B and 19C show an example of a distal portion of a second catheter, such as can be provided by the inner second catheter.

FIGS. 20, 21, 22A, 22B, 23A and 23B disclose examples of systems of a second catheter and a stylet to be inserted into a bore of the second catheter, including various tapering arrangements of and between such components.

### DETAILED DESCRIPTION

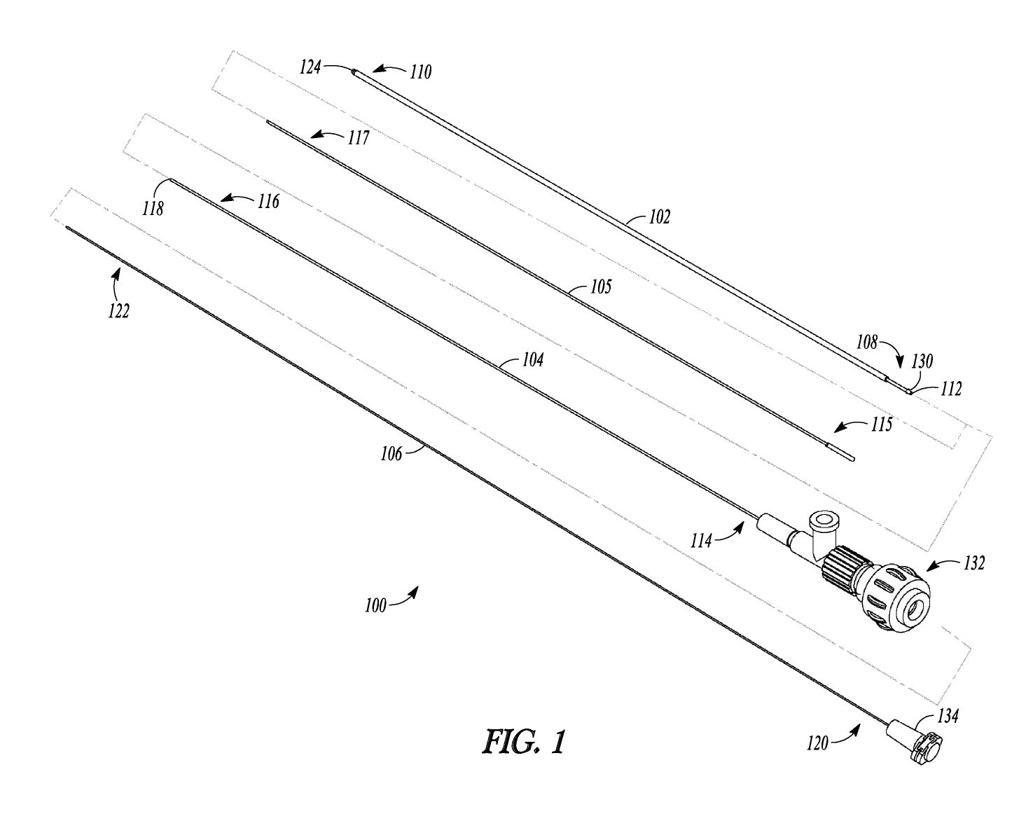

FIG. 1 shows an example of portions of an instrument 100 that can include an outer first catheter 102 body, an inner second catheter 104 body, an obturator or outer first stylet 105, and an obturator or inner second stylet 106, and an example of an insertion arrangement of these components.

In an example, the outer first catheter 102 body can include a proximal portion 108, a distal portion 110, and a longitudinal bore 112 that can extend between the proximal portion 108 of the outer first catheter 102 body and the distal portion 110 of the outer first catheter 102 body. The inner second catheter 104 body can include a proximal portion 114, a distal portion 116, and a longitudinal bore 118 extending between the proximal portion 114 of the inner second catheter 104 body and the distal portion 116 of the inner second catheter 102 body. The first stylet 105 can include a proximal portion 115 and a distal portion 117. The second stylet 106 can include a proximal portion 120 and a distal portion 122. The proximal portion 108 of the outer first catheter body 102 can optionally include a seat 130, such as can be overmolded, overextruded, overshrunk onto or otherwise formed upon a polyimide or polyether ether ketone (PEEK) or other material forming the proximal portion 108 of the outer first catheter body. The proximal portion 114 of the inner second catheter body 104 can optionally include a clamp, collar, or handle 132. The proximal portion 120 of the second stylet 106 can include a clamp, collar, or handle 134.

In an example, the bore 112 of the outer first catheter 102 can be sized and shaped to allow at least a portion of the inner second catheter 104 to be inserted into or through the bore 112, such as to provide enough clearance therebetween to smoothly guide the inner second catheter 104 by the bore 112. The bore 112 of the outer first catheter 102 can be sized and shaped to allow at least a portion of the stainless steel, nitinol or other first stylet 105 to be inserted into or through the bore 112, such as to provide enough clearance therebetween to guide the first stylet 105 by the bore 112. The bore 118 of the inner second catheter 104 can be sized and shaped to allow at least a portion of the stainless steel, nitinol, or other second stylet 106 to be inserted into or through the bore 118, such as to provide enough clearance therebetween to guide the stylet 106 by the bore 118.

While it is possible to use a single catheter for infusion, using two different catheters can help meet multiple different objectives. In an example, the outer first catheter 102 can be more rigid than the inner second catheter 104, for example, such that the outer first catheter 102 can provide steering or guidance for the inner second catheter 104, such as while introducing the instrument 100 into the brain tissue or other target location of the subject. The instrument 100, or one or more portions thereof, can be introduced into the subject via a trajectory guide device, such as using computed tomography (CT), magnetic resonance (MR) imaging, or another imaging modality, such as using an image-guided stereotactic guidance technique.

In an example, introducing the instrument 100 into the brain tissue or other target location of the subject can include inserting the outer first catheter 102 into the subject while the first stylet 105 is located within the bore 112 of the outer first catheter 102, such as with the respective distal ends of the respective distal portions 110 and 117 arranged to be either flush or protruding in a tapered or stepped fashion (e.g., the more interior component, such as the first stylet 105, protruding slightly farther than the more outward component, such as the outer first stylet 102). This can help inhibit or prevent “coring,” which can refer to tissue damage that could otherwise result from tissue being caught in the bore 116 or in the bore 110 during insertion of the instrument 100 into the subject. In an example, introducing the instrument 100 into the brain tissue or other target location of the subject can optionally omit using the first stylet 105 and can include inserting the outer first catheter 102 into the subject while the second stylet 106 is located within the bore 118 of the inner second catheter 104, which, in turn, is located within the bore 112 of the outer first catheter 102, such as with the respective distal ends of the respective distal portions 122, 118, and 110 arranged to be either flush or protruding in a tapered or stepped fashion (e.g., the more interior component, such as the second stylet 106, protruding slightly farther than the next more outward component, such as the inner second catheter 104, which, in turn, can protrude slightly farther than the next more outward component, such as the outer first catheter 102). This can help inhibit or prevent “coring,” such as explained above.

After insertion of the outer first catheter 102 to a target location in the subject, the first stylet 105 (if used during insertion) can be removed. The inner second catheter 104 then can be inserted in place of the first stylet 105, such as with the second stylet 106 already inserted through the bore 118 of the inner second catheter 104, such as to provide rigidity for the inner second catheter 104 during its insertion into the bore 112 of the outer first catheter 102, or to inhibit or prevent tissue coring, such as if the distal end of the inner second catheter 104 is to extend beyond the distal end of the outer first catheter 102. Regardless of whether the second stylet is pre-inserted into the bore 118 of the inner second catheter 104, the bore 118 can be pre-filled with a liquid, such as the liquid infusate, or saline solution, or other biocompatible liquid such as to help avoid introducing air bubbles into the target region of the subject during infusion. In an example, the pre-filling can include drawing the biocompatible liquid into the bore 118 through the distal opening of the bore 118, such as to inhibit or reduce the presence of air bubbles at or near the distal opening of the bore.

After insertion of the inner second catheter 104 to a desired target location within the subject, the second stylet 106 can be partially or completely removed from the bore 118, such as at the proximal portion 114 of the inner second catheter 104. Then, infusion can then be performed to deliver (e.g., via the bore 118 of the inner second catheter 104) a liquid or other fluid substance to a target location within the subject, such as with the inner second catheter 104 still located within the bore 112 of the outer first catheter 102. Then, either both of the outer first catheter 102 and the inner second catheter 104 can be removed together, or the inner second catheter 104 can be removed while the outer first catheter 102 can be left in place (or vice-versa), such as to allow later re-insertion or reuse of the inner second catheter 104 for further infusion at a later time. Such later infusion can be carried out over an acute period of time (e.g., while the patient is hospitalized) or even over a chronic period of time (e.g., which can include time periods between hospitalizations or doctor visits or a combination thereof).

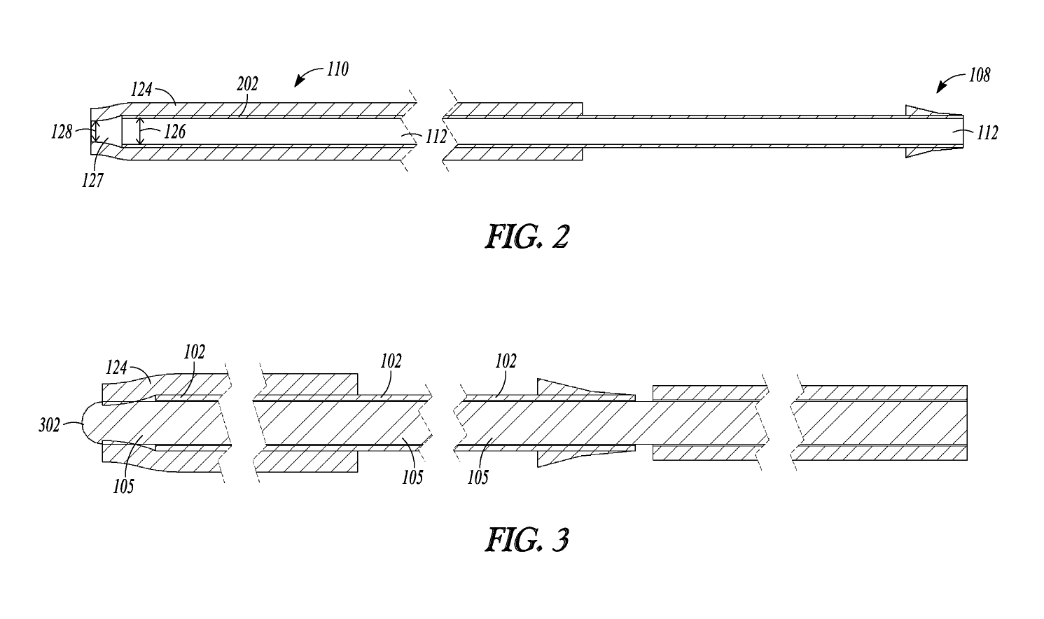

In FIG. 1, an elastically deformable first seal 124 can be located at the distal portion 110 of the outer first catheter 102 body, such as shown in more detail in FIG. 2. FIG. 2 shows a cross-sectional view of an example of the outer first catheter 102, including the distal portion 110 of the outer first catheter 102. An elastically deformable first seal 124 can be located at the distal portion 110 of the outer first catheter 102 body. In an example, the outer first catheter body 102 can be formed from a (e.g., harder) different material (e.g., polyimide or polyether ether ketone (PEEK)) than that of the first seal 124 (e.g., silicone or polyurethane). The first seal 124 can be configured to elastically deformably and conformally encompass an outer periphery of an instrument, such as the first stylet 105 or the inner second catheter 104, when such instrument has been inserted through the bore 112 of the outer first catheter 108 body such that the distal end of such instrument extends to a distal end or even out of the lumen 112 of the distal portion 102 of the outer first catheter 102.

In an example, the elastically deformable first seal 124 can be more flexible than a more proximal portion of the outer first catheter body 102, such as the distal portion 110 of the outer first catheter body 102 providing a cannula 202 having an inner circumferential periphery defining the bore 112. The first seal 124 can define an inner circumferential periphery (e.g., represented by a lateral inner dimension 128) that, in a first configuration, is laterally smaller than the inner circumferential periphery (e.g., represented by a lateral inner dimension 126) of a more proximal portion of the bore 112 to at least partially occlude the bore 112 at the distal portion 110 of the outer first catheter body 102 and, in a second configuration, the inner circumferential periphery (e.g., represented by a lateral inner dimension 128) of the first seal 124 is expandable relative to the first configuration.

In the example shown in FIG. 2, the first seal 124 at least partially occludes the bore 112 at the distal end of the distal portion 110 of the outer first catheter body 102 in the absence of the first stylet 105, the inner second catheter 104, or other instrument having been inserted through the bore 112 of the outer first catheter 102 body such that such instrument extends to the distal end or out of the distal portion 110 of the outer first catheter 102 body. In an example, such partial occlusion can be present when the inner lateral dimension 128 of a portion of a lumen 127 of the first seal 124 is less than the inner lateral dimension 126 of the bore 112 at the distal end of the distal portion 110 of the outer first catheter body 102, such as in a first configuration, e.g., when the inner second catheter 104 or other instrument has not been inserted through the lumen 127. In an example, the inner lateral dimension 128 of the lumen 127 of the first seal 124 can equal or exceed the outer lateral dimension 123 of the first stylet 105 or the second stylet 106, such as to allow the first stylet 105 or the second stylet 106 to extend with its distal end flush with or beyond a distal end of the first seal 124, such as to help inhibit or prevent tissue coring. FIG. 3 shows an example of the distal portion 110 of the outer first catheter 102 with a rounded or blunt distal end of the first stylet 105 extending slightly beyond the distal end of the first seal 124, such as by a distance that is approximately or exactly equal to a radius of curvature of a rounded distal end of the first stylet 105, such as to help inhibit or prevent tissue coring.

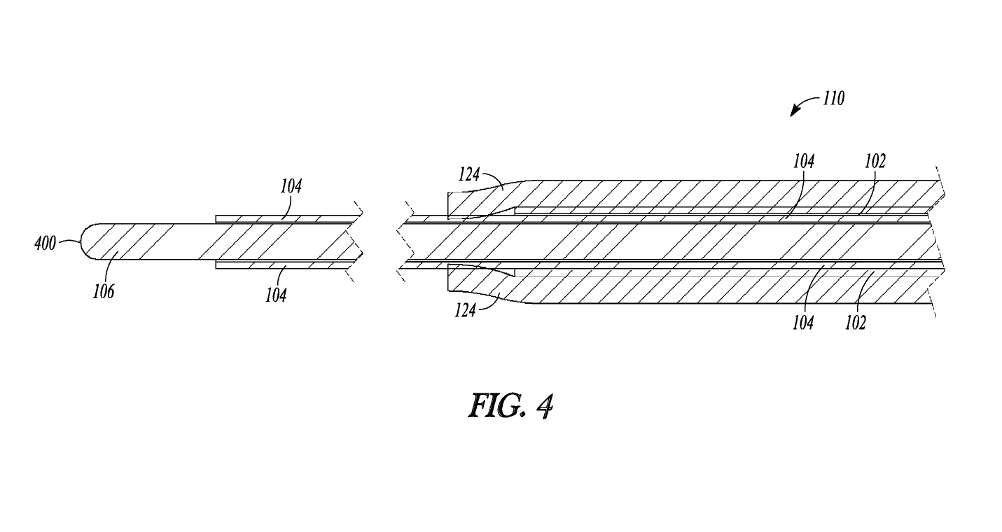

FIG. 4 shows a cross-sectional view of an example of the distal portion 110 of the outer first catheter 102. The inner second catheter 104 can be inserted through the bore 112 of the outer first catheter 102 body such that the distal end of the inner second catheter 104 or other instrument can extend out of the lumen 112 of the distal portion 110 of the outer first catheter 102. Before insertion of the inner second catheter 104 body into the bore 112 of the outer first catheter 102 body, the second stylet 106 can be pre-inserted through the bore 118 of the inner second catheter 104 body, such that rounded or blunt end 400 of the second stylet 106 can extend beyond the distal end of the inner second catheter 104. This can help provide rigidity to the inner second catheter 104 during insertion or can help inhibit or prevent tissue coring. In an example, the distal end of the second stylet 106 can be inserted beyond the distal end of the inner second catheter 104 by an amount beyond flush that is sufficient to accommodate a tolerance in an insertion limit such as can be established by a stop or the like at the proximal end 120 of the second stylet 106. When the second stylet 106 is retracted, a tissue void may remain, which can provide a volume into which infusate can be delivered.

In the example of FIG. 4, the first seal 124 is demonstrated to elastically deformably and conformally encompass the outer periphery of an instrument, such as the inner second catheter 104, when the inner second catheter 104 (or other instrument) has been inserted through the bore 112 of the outer first catheter 102 body such that the distal end of the inner second catheter 104 or other instrument extends to a distal end or even out of the lumen 112 of the distal portion 110 of the outer first catheter 102. In this example of a “second configuration,” with the inner second catheter 104 protruding out from the lumen 127 of the first seal 124, the inner lateral dimension 128 of the lumen 127 of the first seal 124 has expanded to accommodate such insertion of the inner second catheter 104 therethrough. The first seal 124 can elastically deformably and conformally encompass the outer periphery of the inner second catheter 104. This can help inhibit or prevent infusate leaving the bore 118 at the distal end of the distal portion of the inner second catheter 104 (e.g., when the second stylet 106 has been partially or completely withdrawn from the bore 118) from wicking or otherwise flowing backward (reflux) into the clearance space between the outer periphery of the inner second catheter 104 and the inner periphery of the outer first catheter 102. Such reflux can lead to waste or inappropriate dosing.

The first seal 124 can optionally be configured to inhibit backflow of infusate into the space between outside of the outer first catheter body 102 along a tract through tissue through which the outer first catheter body 102 has been inserted. In an example, the first seal 124 can be integrally formed with a boot or jacket that encompasses an outer portion of at least a portion of the outer first catheter body 102, such as shown in FIG. 2, in which the first seal 124 extends backward from the distal end of the outer first catheter body 102 at least some distance toward the proximal end 108 of the outer first catheter body 102.

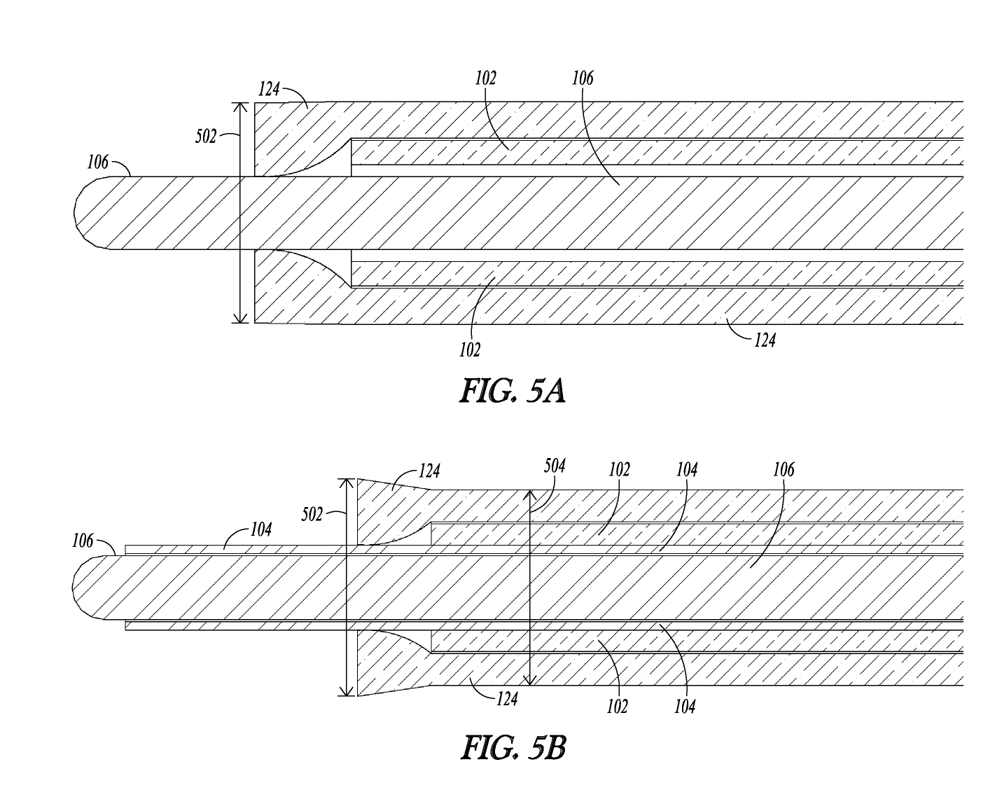

FIGS. 5A and 5B show an example in which an outer dimension 502 of the outer periphery of the first seal 124 can be expanded from that of a first configuration, such as shown in FIG. 5A, (e.g., by insertion of an instrument such as the inner second catheter 104 therethrough) such as to flare out to be laterally wider, in a second configuration, than a proximally adjacent outer dimension 504 of the distal portion 110 of the outer first catheter 102 body or of the first seal 124 such as by an amount that is sufficient to help inhibit backflow of infusate outside of the outer first catheter 102 body along a tract through tissue through which the outer first catheter body 102 passes.

Additionally or alternatively, an inflatable balloon can be located about the outer periphery of the first seal 124 or the distal portion 110 of the outer first catheter 102 body, which balloon can be inflated to help inhibit or prevent infusate reflux outside of the outer first catheter body 102 such as along a tract through tissue through which the outer first catheter body 102 passes.

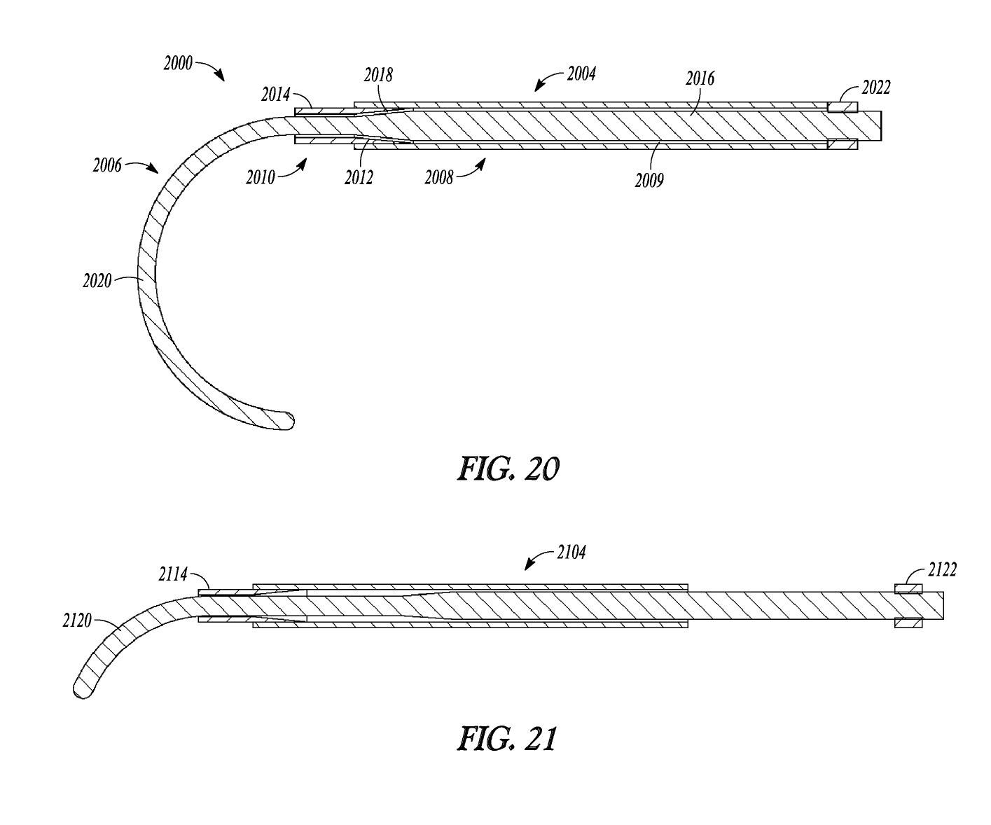

Additionally or alternatively, the first stylet 105, the second stylet 106, or the inner second catheter 104 can be provided with a circumferential groove, which can be located so as to be set back from the distal end of such component, such as by a desired amount (which may depend on relative taperings, such as described below, such as with respect to FIGS. 20, 21, 22A, 22B, 23A, and 23B, and elsewhere in this document. The groove can be configured such that when such component is retracted in a proximal direction, the distal opening of the first seal 124 can catch within the groove, thereby pulling proximally the “caught” distal opening of the first seal 124, thereby causing outward lateral distension of a slightly more proximal portion of the first seal 124, which can also help inhibit or prevent infusate reflux around the outer first catheter 102 along the tract through the tissue created by the outer first catheter 102.

In an example, the first seal 124 can be sized and shaped to present a substantially laterally extending distal edge forming a substantially orthogonal angle to a distal longitudinal direction of the outer first catheter 102 body such as to help inhibit or prevent backflow of infusate outside of the outer first catheter 102 body along a tract through tissue through which the outer first catheter 102 body has been inserted, such as shown in FIGS. 3 and 4.

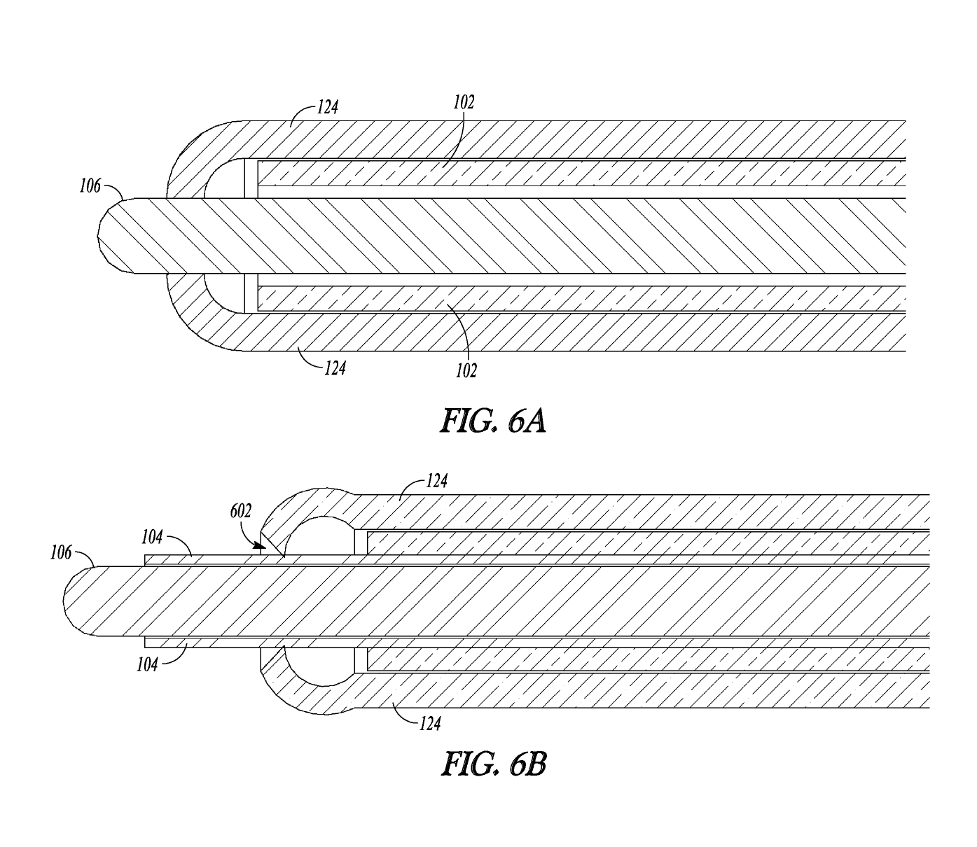

In an example, the first seal 124 can be sized and shaped to present a substantially laterally extending distal edge forming an acute angle 602 to a distal longitudinal direction of the outer first catheter 102 body such as to help inhibit or prevent backflow of infusate outside of the outer first catheter 102 body along a tract through tissue through which the outer first catheter 102 body passes, such as shown in FIG. 6B. In the example of FIGS. 6A-6B, the distal edge of the first seal 124 can be actuated from a substantially orthogonal angle in a first configuration, such as shown in FIG. 6A, to the acute angle 602 in a second configuration, such as shown in FIG. 6B, such as by the inner second catheter 104 body being inserted through the lumen 127 of the first seal 124.

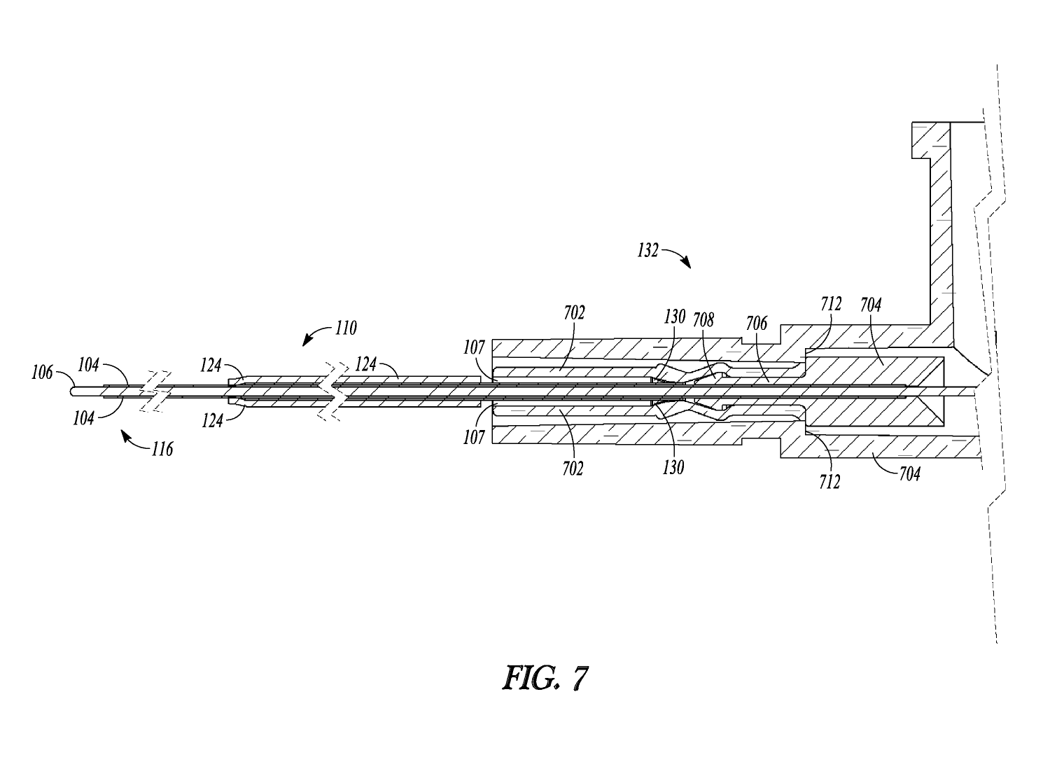

FIG. 7 shows an example of a proximal elastically deformable (e.g., silicone or polyurethane) second seal 702 that can additionally or alternatively (to the first seal 124) be provided, such as to help inhibit or prevent infusate reflux into the clearance space between (1) the outer periphery of the inner second catheter 104 and (2) the inner periphery of the outer first catheter 102. The elastically deformable second seal 702 can be located at or toward the proximal portion 108, 114 of one of the outer first catheter 102 body or the inner second catheter 104 body. The second seal 702 can be configured to be elastically deformable such as to conformally encompass an outer periphery of a proximal portion of the other one of the outer first catheter body 102 or the inner second catheter body 104 when at least a portion of the inner second catheter 104 has been inserted into or through the bore 112 of the outer first catheter 102 body. The other one of the outer first catheter body 102 or the inner second catheter body 104 can include a seat 130. The seat 130 can protrude laterally outward from and peripherally about the proximal portion 108, 114 of the other one of the outer first catheter 102 body or the inner second catheter 104 body. The seat 130 can be sized and shaped to be positioned against or within or to be encompassed by the second seal 702, such as shown in the example of FIG. 7. This can help inhibit or prevent infusate reflux into the clearance space between the outer periphery of the inner second catheter 104 and the inner periphery of the bore 112 of the outer first catheter 102.

In the example shown in FIG. 7, the second seal 702 can be located on the proximal end 114 of the inner second catheter 104 body and the seat 130 can be located on the proximal end 108 of the outer first catheter 102 body. Alternatively, the second seal 702 can be located on the proximal end 108 of the outer first catheter 102 body and the seat 130 can be located on the proximal end 114 of the inner second catheter body 104. In an example, the second seal 702 can be carried within a clamp, collar, or handle 132, such as shown in FIG. 7. In an example, the handle 132 can include a polycarbonate or other T-connector 704 (a Y-connector can be substituted for the T-connector throughout the several instances of a T-connector described herein). The seal 702 can be mounted within the T-connector, such as onto a longitudinally-oriented spindle 706 or other member. The seal 702 can be mounted onto the spindle 706 so as to provide a cylindrical or like receptacle 707. The receptacle 707 can open in a distal direction, such as to receive all or a portion of the seat 130 therein. The spindle 706 can include a laterally outwardly protruding flare, flange, barb, seat, or other retainer 708, such as at a distal end of the spindle 706 within the receptacle. The retainer 708 can help secure the second seal 702 to the spindle 706, such as to allow insertion of the seat 130 well into the receptacle of the second seal 702, or retraction of the seat 130 therefrom. A proximal portion 710 of the spindle 707 can flare laterally outward, so as to be adhered or otherwise secured against a flange 712 within the T-connector 704. In an example, the first seal 124 can extend in a boot or jacket about the outer circumferential periphery of the outer first catheter 102, such as for a substantial distance to the proximal portion 108 of the outer first catheter 102. In this way, the proximal edge of the first seal 124 can act as an insertion depth stop for the seat 130 being inserted into the receptacle 707 of the second seal 702. When the proximal edge of the first seal 124 abuts the distal edge of the second seal 702, the user can know that the seat 130 is properly located at a desired location within the second seal 702.

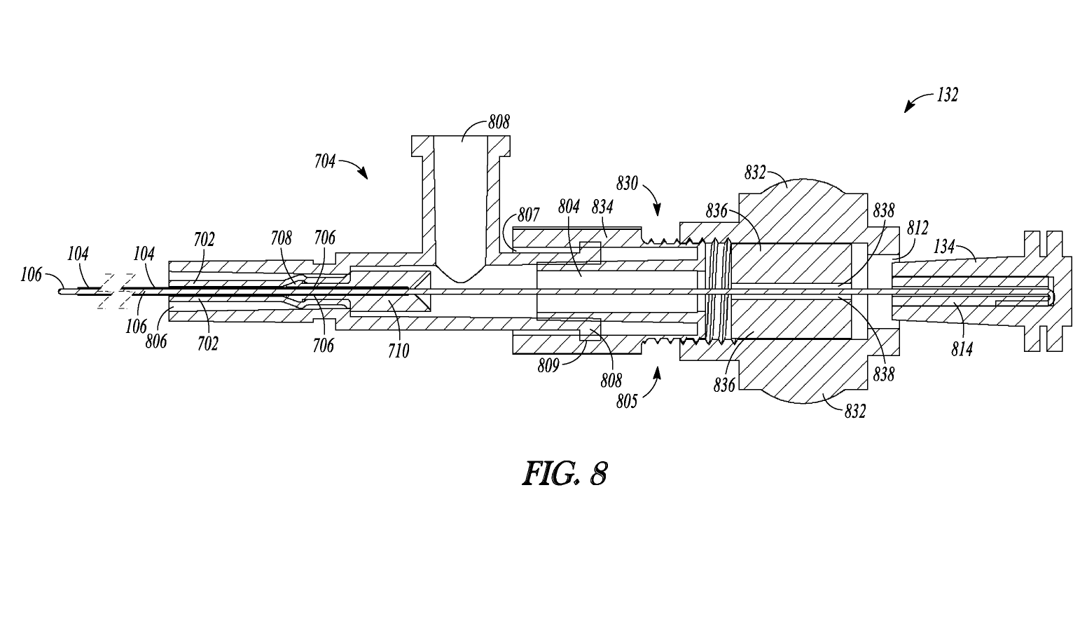

FIG. 8 shows an example of portions of the handle 132. The handle 132 can include a Y-connector or a T-connector 704 body, which can include a proximal port 804, a distal port 806, and a lateral side port 808, each of which can be interconnected with the others within the T-connector 704 body, such as to allow fluid communication therebetween.

The distal port 806 can be sized or shaped or otherwise configured to receive, engage, snap-fit, thread onto, or be coupled to the proximal end of the inner second catheter 204. This can allow fluid communication between the T-connector 704 body and the inner second catheter 104 via the distal port 806, such as to allow infusion at a target location at or near a distal portion 116 of the inner second catheter 104. A secure fit or seal with the bore 118 of the inner second catheter 204 can be included. This can help ensure that any liquid infusate within the T-connector 704 body leaves the distal port 806 only via the lumen 118 of the inner second catheter 104. The seal 702 can seal between the outer circumferential periphery of the inner second catheter 104 and the inner circumferential periphery of the bore 112 of the outer first catheter 102, such as described above, such as to inhibit or prevent wicking capillary action or other reflux of infusate therebetween at the distal end of the instrument 100. The seal 702 can be provided in addition to or as an alternative to the seal 124, such as for such reflux inhibition. Additionally or alternatively, a hydrophobic coating or gel can be provided on one or more of the outer circumferential circumferential periphery surface of the inner second catheter 104 or the inner circumferential periphery surface of the outer first catheter 102, such as at least near their respective distal ends, or extending longitudinally along a substantial portion or even the entire length to their respective proximal portions. This can help adjust the surface tension characteristics, such as to inhibit or prevent wicking capillary action of infusate, blood, cerebrospinal fluid (CSF) or the like.

The proximal port 804 of the T-connector 704 can be configured to receive, engage, snap-fit, thread onto, or be coupled to a distal port 807 of a clamp, collar, or head 805. In an example, a Luer taper connection can include one or more of polycarbonate, polyisoprene, latex rubber, or acrylonitrile-butadiene-styrene (ABS) or one or more other materials, and can be provided between the proximal port 804 of the T-connector 704 and the distal port 807 of the head 805. The Luer taper connection can optionally include a thread 808 on one of the proximal port 804 of the T-connector 704 or the distal port 807 of the head 805, which can be configured to thread into and engage a corresponding thread 809 on the other of the proximal port 804 of the T-connector 704 or the distal port 807 of the head 805. A proximal port 812 of the head 805 can be tapered or otherwise configured to receive and engage the handle 134 or other fitting at the proximal portion 120 of the second stylet 106. The handle 134 can be secured to the proximal end of the second stylet 106, such as by bending a stainless steel or nitinol wire or other material forming the second stylet around a tube 814 carried within a longitudinal center receptacle of the rigid or other handle 134. The handle 134 can provide a seal or stopper to seal the proximal port 812 of the head 805, such as to allow infusion of a liquid introduced at the side port 808 of the T-connector 704 to be directed toward the distal port 806 of the T-connector 704 and through the bore 118 of the inner second catheter 104 to the distal portion 118 of the inner second catheter 104 for delivery to a target location within a subject. In an example, this can include coupling the side port 808 to an infusion pump or other infusion source, such as via a tube or the like.

The head 805 can include a compression element, such as a set screw, a Luer taper stopper, a Tuohy-Borst connector 830, or the like, which can be configured to be capable of being user-actuated such as to selectably press against the proximal portion 120 of the second stylet 106, such as to secure the proximal portion 120 of the second stylet 106 to the head 805 and, in turn, to the T-connector 704 body, such as at or near the proximal port 804 of the T-connector 704.

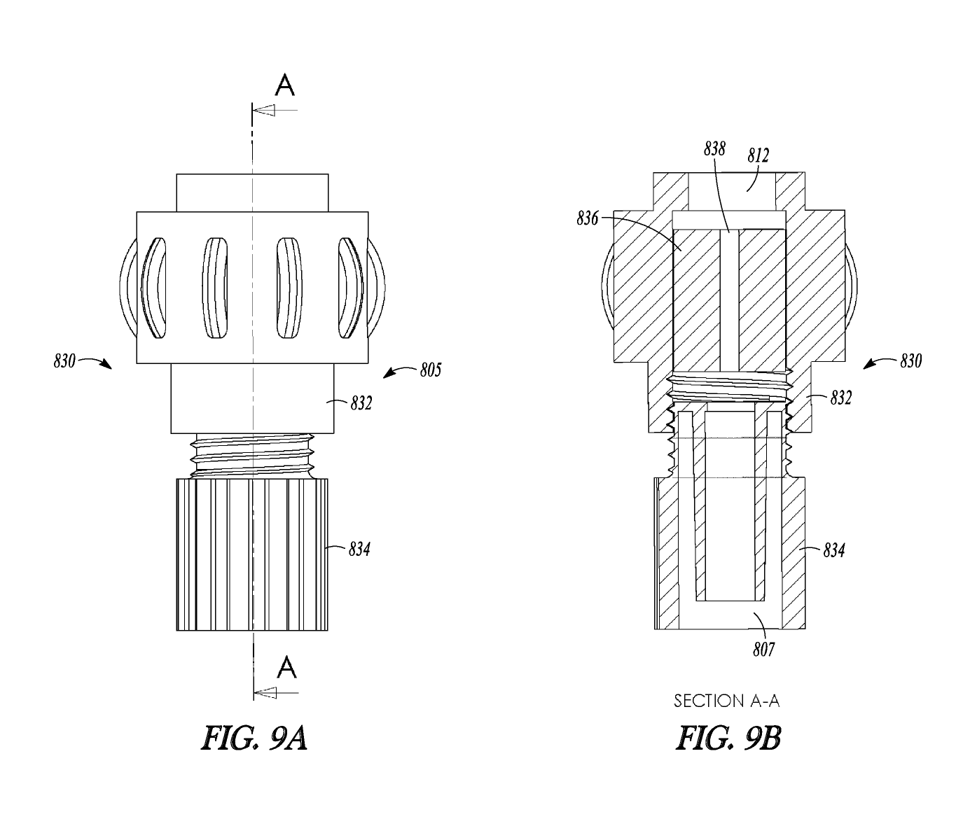

In an example, the compression element of the head 805 can be provided by a Tuohy-Borst connector 830, an example of which is shown in further detail in FIGS. 9A and 9B. The Tuohy-Borst connector 830 can include twist-together or other tubular couplings, such as a first coupling 832 and a second coupling 834, which can be made of fluoropolymer or polycarbonate. The first coupling 832 and the second coupling 834 can be threaded or otherwise drawn together longitudinally, such as to longitudinally squeeze a compressible tubular silicone or other stopper 836 that includes a lumen 838 through which the second stylet 106 can be passed. Such longitudinal squeezing of the compressible tubular stopper 836 can constrict lumen 838 of the tubular stopper 836, such as to secure or seal the proximal portion 120 of the second stylet 106 passing through the lumen 838 of the stopper 836 of the Tuohy-Borst connector 830.

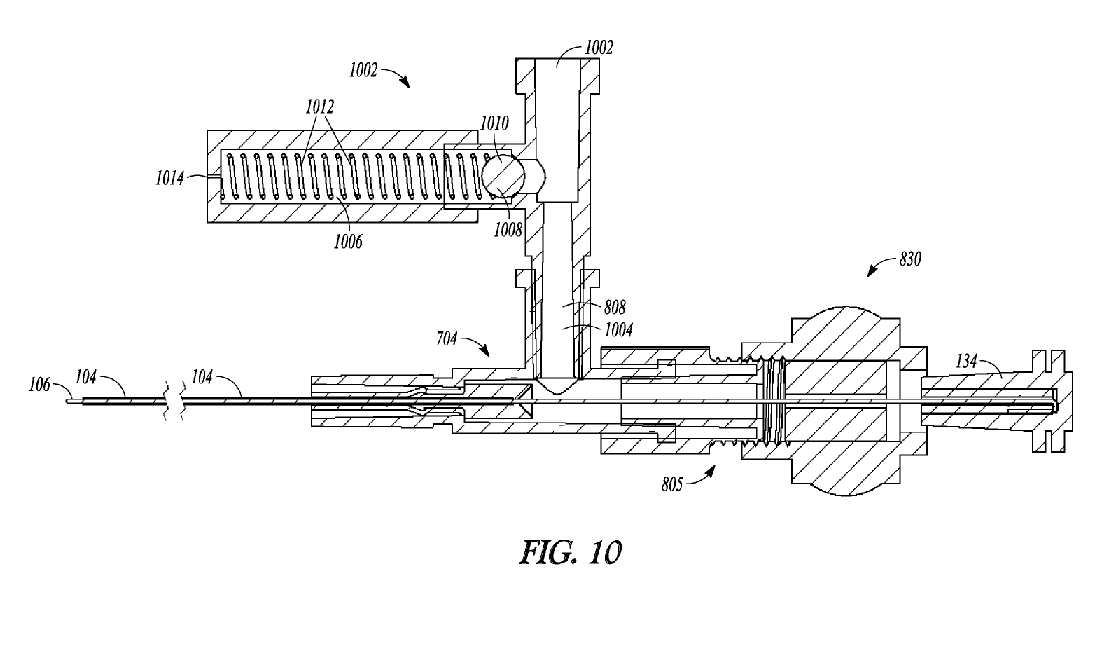

FIG. 10 shows an example of a pressure-relief valve assembly 1002, such as can be configured to be received, engaged, snap-fitted, threaded onto, or otherwise coupled to the side port 808 of the T-connector 704, such as via a threaded or other Luer taper connection. The pressure-relief valve assembly 1002 can be configured to be in fluid communication with the longitudinal bore 118 of the inner second catheter 104. The pressure relief valve assembly 1002 can be configured to open in response to a particular fluid pressure, such as a fluid pressure within the longitudinal bore 118 of the inner second catheter 104 meeting or exceeding a specified maximum pressure threshold value. In response, the pressure relief valve assembly 1002 can vent infusate away from the distal portion 116 of the inner second catheter 104, via the bore 118, such as into the pressure-relief valve assembly 1002. This can help avoid damage to sensitive brain tissue that can be located near the distal end of the distal portion 116 of the inner second catheter 104, such as might otherwise be caused by excess infusate fluid pressure.

In an example, the pressure relief valve assembly 1002 can include an input port 1002 that can be coupled to an infusion pump or other infusion source, such as via a tube or the like. The input port 1002 can be in fluid communication with an output port 1004 of the pressure relief valve assembly 1002. The output port 1004 of the pressure relief valve assembly 1002 can be configured to be in fluid communication with the longitudinal bore 118 of the inner second catheter 104, such as via the side port 808 of the T-connector 704. The pressure relief valve assembly 1002 can also include a pressure-relief port or reservoir 1006. The pressure relief valve assembly 1002 can include a ball valve or other valve 1008. The valve 1008 can be configured to isolate or seal the pressure-relief port or reservoir 1006 from the input port 1002 and from the output port 1004 when the fluid pressure within the longitudinal bore 118 of the inner second catheter 104 is less than the specified maximum pressure threshold value. The valve 1008 can be configured to vent fluid from the input port 1002 or the output port 1004 to the pressure-relief port or reservoir 1006 in response to the fluid pressure within the longitudinal bore 118 of the inner second catheter 104 meeting or exceeding the specified maximum pressure threshold value. In an example, the valve 1008 can include an occlusion device, such as a ball 1010, and a biasing device, such as a coil or other spring 1012. The spring 1012 can bias the ball 1010. In an example, the specified maximum pressure threshold value can depend at least in part on a spring constant characteristic of the spring 1012. In an example, the pressure-relief port or reservoir 1006 can include an exit passage 1014. In an example, the exit passage 1014 can be configured with a membrane sealing the exit passage 1014. The membrane can be gas-permeable and liquid-inhibiting or liquid-impermeable, such as to allow passage of air and water vapor out of the pressure-relief port or reservoir 1006 via the exit passage 1014, but to inhibit or prevent passage of liquid fluid out of the pressure-relief port or reservoir 1006 via the exit passage 1014. In an example, the “breathable” membrane material can include a flashspun high-density polyethylene fiber, such as Tyvek® from E.I. du Pont de Nemours and Co., of Wilmington, Del., U.S.A.

The pressure threshold value can be adjusted, such as by changing the resilience of the spring 1012 to the desired setting. In an example, such changing can be user-provided, such as to allow user-adjustment of the pressure threshold. For example, the user can be allowed to select or adjust a thumbscrew that can alter the length of the spring 1102, such as to change the displacement of the spring 1838, such as to pre-load the biasing force provided by the spring 1838 to a user-adjustable value. User selection or adjustment of any other valve characteristic can similarly be provided.

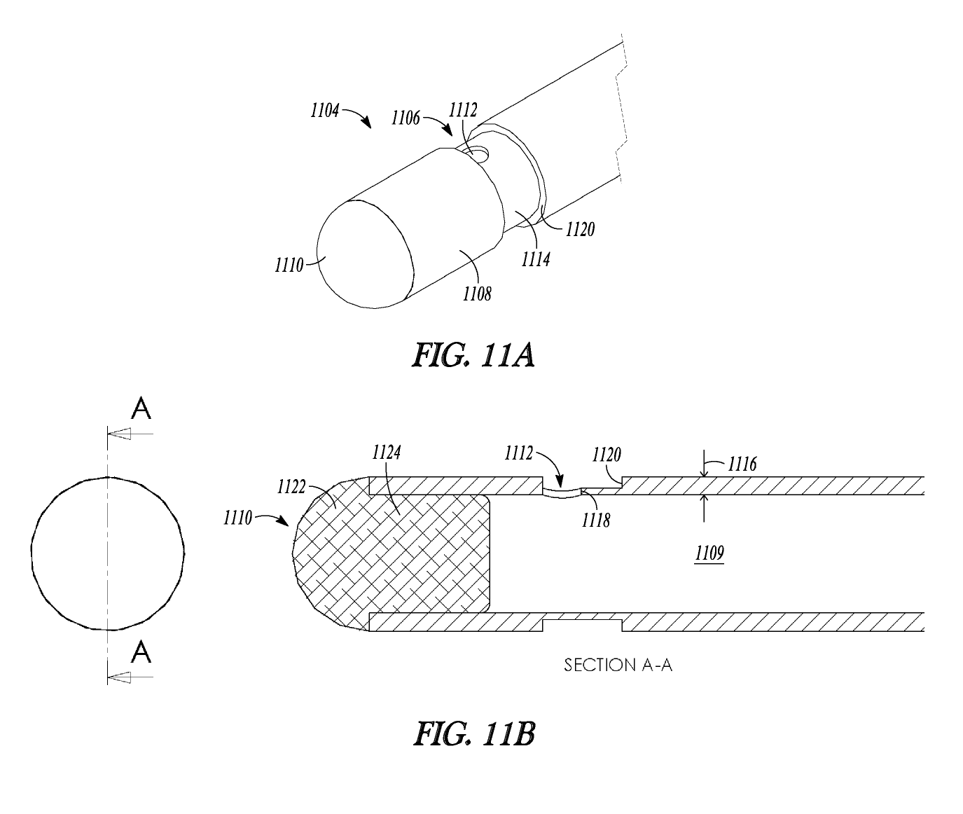

FIGS. 11A and 11B show an example of a distal portion 116 of an inner second catheter 104. FIG. 11B shows a cross-sectional view of the distal portion 116 of the inner second catheter 104 that is shown in perspective in FIG. 11A.

The inner second catheter 104 can be sized or shaped or otherwise configured for insertion into or through the bore 112 of the outer first catheter 102. For example, the outer periphery of the inner second catheter 104 can be chosen such that it can slidably engage the bore 112 of the outer first catheter 102. The diameter or similar lateral dimension of the outer periphery of the inner second catheter 104 can be chosen to be a value that can be up to 99.99% of the value of the diameter or similar lateral dimension of the inner periphery of the bore 112 of the outer first catheter 102. These lateral dimensions can be chosen to reduce or minimize the cross-sectional area available between the outer first catheter 102 and the inner second catheter 104, such as to reduce or minimize wicking or other fluid flow, such as reflux of an infusate between the outer first catheter 102 and the inner second catheter 104. The distal portion 116 of the inner second catheter 104 can include a catheter body 1108 and a distal tip 1110, which can be separate components that are attached to each other, or can be integrally connected to each other or can be made from the same material. The proximal portion 114 of the inner second catheter 104 can be designed according to one of the examples shown elsewhere in this document, such as in FIGS. 1-8, such as to provide a working interaction of the inner second catheter 104 with a clamp, collar or handle such as exemplified in FIG. 1, or to provide a working interaction with the outer first catheter 102.

The catheter body 1108 can comprise a distal portion such as shown in FIG. 11A and a proximal portion that is not shown in FIG. 11A. As can be seen in the cross-sectional view of FIG. 11B, the catheter body 1108 can include a bore 118 that can extend from the proximal portion 114 to the distal portion 118. The bore 118 can be used for channeling infusate from the proximal portion 114 to the distal portion 118. In an example, the catheter body 1108 can be made from polyether-ether-ketone (PEEK), polyimide, or another rigid material, such as described in the examples of the outer first catheter 102 or the inner second catheter 104. The body 1108 can comprise a through-opening 1112 connecting the outer periphery of the body 1108 with the bore 118. This opening 1112 can provide an infusion port that can be used to infuse a liquid or other flowable substance into nearby or surrounding brain or other tissue. The opening 1112 can be shaped in a circle, oval, or multi-edged shape such as a rectangle. Resulting corners of a multi-edged shape can be rounded. The edges of the opening 1112 in a direction perpendicular to the axis of the catheter body 1108 are shown in FIG. 11A as being essentially straight, in an example, but can be shaped to taper, bevel, or chamfer, such as resulting in a wider cross-section of the opening 1112 along the outer periphery of the catheter body 1108. However, the taper of the edges of the opening 1112 can be reversed. The opening 1112 through the catheter body 1108 can be drilled, etched, molded, or stamped or can otherwise be formed by any other method. The opening 1112, such as shown, can be shaped to have a smaller cross-sectional area than the cross-sectional area of the bore 118 of the inner second catheter 104 providing the catheter body 1108, but the size of the opening 1112 can be chosen differently. Even though the illustrated example shows only a single opening 1112, one or more further (e.g., similarly or differently shaped) openings can be introduced into the catheter body 1108, such as either in the longitudinal direction of the catheter body 1108 or along the circumference of the catheter body 1108. Such one or more further openings can be used as an infusion port or other liquid port, such as to transport infusate or other liquid.

The opening 1112 can be situated in a recessed portion 1114 of the catheter body 1108. The recessed portion 1114 can have a wall thickness 1118 that can be thinner than the wall thickness 1116 that is adjacent to or surrounding the recessed portion 1114. In an example such as shown in FIG. 11A, the recessed portion 1114 can include a circumferential ring or groove or indentation around the circumference of the outer periphery of the catheter body 1108. However, the recessed portion 1114 can be shaped differently, for example in an disk shape, rectangular shape or oval shape, such as where the shape has an exposed surface area that is greater than the area of opening 1112, such as to fully encompass the opening 1112. Regardless of such variation in shape, the recessed portion 1114 can be shaped to inhibit or avoid skiving of nearby or surrounding tissue. The cross-section of the outer periphery of the catheter body 1108 adjacent (e.g., proximal or distal) to the recessed portion 1114 can be enough larger than the cross-section of outer periphery of the recessed portion 1114 such that brain or other adjacent tissue skimming over the recessed portion 1114 is not skived or pared by the at least one opening 1112, which can lead to tissue damage or skived tissue clogging the opening 1112. Instead, such adjacent tissue can skim over the opening 1114 without being skived. To similarly decrease the chance of skiving tissue, the leading (e.g., distal) or trailing (e.g., proximal) edges of the recessed portion 1114, such as the trailing/proximal edge 1120, which are shown as being perpendicular to the longitudinal direction of the catheter body 1108, can be arranged under a different angle, or can have rounded, multifaceted, or otherwise smoothed edges. If the angle shown is defined as 90°, the angle (facing inward toward the recessed portion 1114) of the edges may be chosen to be obtuse, such as between 90° and 1800°, or between 120° and 160°. In an example, the wall-thinning in the recessed portion 1114 (which can be shaped as shown or as otherwise described herein) can be continuous instead of the illustrated discrete step, for example, the wall thickness 1118 can continuously decrease from the wall thickness 1116 to the lesser thickness at the opening 1112.

In an example, the distal tip 1110 can be mushroom shaped, such as to slidingly or fixedly engage the inner periphery of the bore 1109 of the catheter body 1108, such as to allow at least a portion of the distal tip 1110 to be extended out from or retracted into the bore 1109 of the catheter body 1108. This can be accomplished by using the second stylet 106 within the bore 1109 of the catheter body 1108 to push out the distal tip 1110. Optionally, a distal tip of the second stylet 106 or other similar stylet can be provided with a hook or other engagement device to engage a corresponding feature on the distal tip 1110 to retract the distal tip 1110, if desired. The distal tip 1110 can include a flange or a key, such as to ride within a slot in the inner circumferential periphery of the bore 1109 of the catheter body 1108, such as to limit the extent to which the distal tip 1110 can be distally extended, such as to provide a “captured” distal tip 1110 that cannot become disengaged from the catheter body 1108 by overextension.

The distal tip 1110 can include a rounded, multifaceted, beveled, chamfered, or tapered tip 1122, such as can be shaped to inhibit or avoid coring or other damage of tissue when the inner second catheter 104 providing the catheter body 1108 is advanced through brain or other tissue in a distal direction. The tip 1122 shape can be different, such as, for example, being cylindrical or parabolic-shaped. In an example, at least a portion of the distal tip 1110 can be made from rubber, silicone, polyurethane, or from one or more other biocompatible materials. The rounded tip 1122 can have the shape of a section of a sphere, which can have a radius similar to the radius of the circumferential outer periphery of the catheter body 1108. But the radius can be chosen smaller or larger in relation to the radius of the catheter body 1108. The stem 1124 and the rounded tip 1122 can be integrally made such as from a block or other shape of the same material, but can be separate parts of separate (e.g., same or different) materials. The stem 1124 can be shaped and sized or otherwise configured such that it does not cover the opening 1112. In another example, the distal tip 1110 can be sized or shaped or otherwise configured to cover the opening 1112, and the distal tip 1100 can include a passage or cavity that can fluidly connect the opening 1112 of the catheter body 1108 with the bore 1109 of the catheter body 1108. The distal tip 1100 or the catheter body 1108 can be coated with a hydrophobic coating or a gel. Such coating may help inhibit or avoid reflux of infusate or other liquid.

As an illustrative example of dimensions, the outer first catheter 102 can have an outer diameter of approximately 1.5 millimeters, the inner second catheter 104 can have an outer diameter of about 0.6 millimeters, the opening 1112 can have a diameter of about 0.127 millimeters, and the depth of the recessed portion 1114 can be about 0.127 millimeters, and the band width of the recessed portion 1114 can be about 890 millimeters, and opening 1112 can be placed at the distal edge of the recessed portion 1114 such as to allow the nearby tissue to skim over the opening 1112 without skiving.

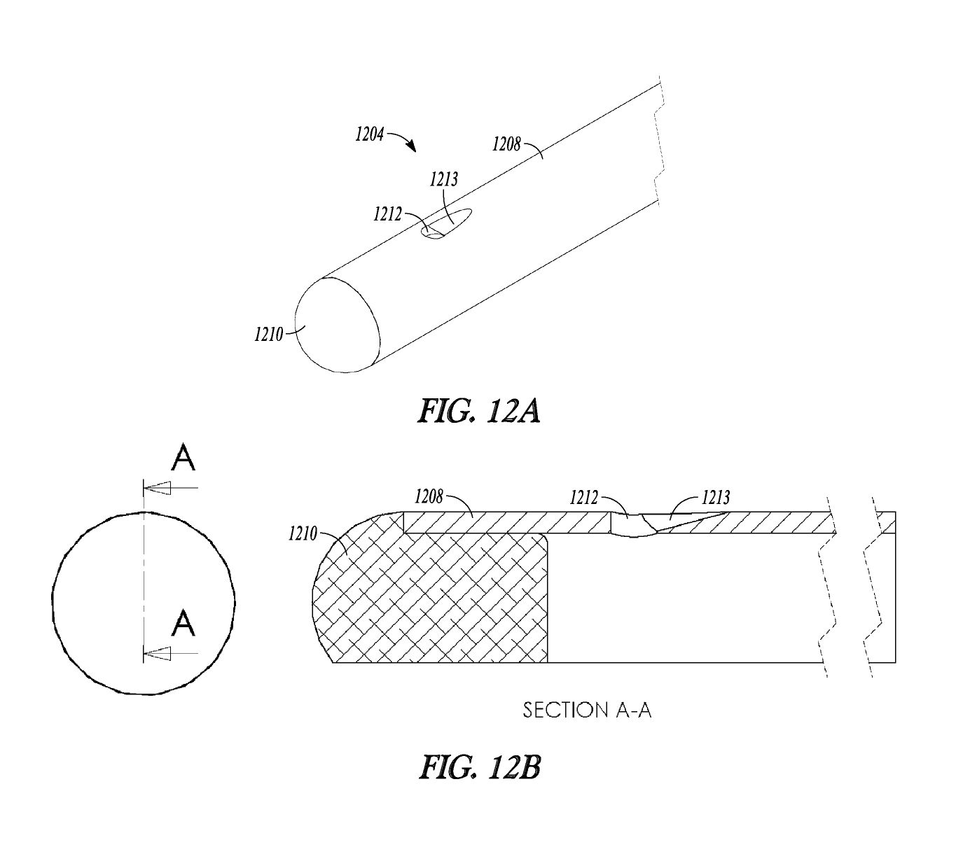

FIGS. 12A and 12B show an example of a distal portion 1206 of an example of a catheter body 1208, such as can be provided by an inner second catheter 104. This example can differ from (or be combined with) the example shown in FIGS. 11A and 11B or elsewhere in that it can provide an opening 1212 that can be differently formed into the catheter body 1208. The catheter body 1208 can omit or include the recessed portion 1114 or circumferential groove (e.g., such as shown in FIG. 11) into which the opening 1212 can be formed. Such a recessed portion 1114 as described in FIG. 11 can be incorporated in other examples of a catheter body shown or described in this document.

The opening 1212 can include a scallop-shaped or other tapered, beveled, or chamfered portion 1213 that can extend in the proximal direction, as shown, or in the distal direction, or in any other direction along the outer periphery of the catheter body 1208. The scallop-shaped tapered, beveled, or chamfered portion 1213 as seen in the cross-sectional view of FIG. 12B can taper in the proximal direction such that the wall of the catheter body 1208 can continuously change its thickness along the scallop-shaped tapered portion 1213 and the cross-sectional area of the opening 1212 can vary in a direction that is perpendicular to the longitudinal direction of the catheter body 1208. The scallop-shaped tapered portion 1213 can be curved, such as shown in FIG. 12A, such as in a direction that is perpendicular to the proximal direction such as to avoid skiving of tissue when the catheter body 1208 is moved in a distal direction. The scallop-shaped portion in connection with the opening 1212 is only one example of a varying cross-sectional area.

The distal tip 1210 can be comparable to distal tip 1110. Examples of further details regarding possible shapes, sizes, materials of the catheter body, the opening, the thin-walled or other recessed portion 1114 or the distal tip are described in connection with FIGS. 11A and 11B.

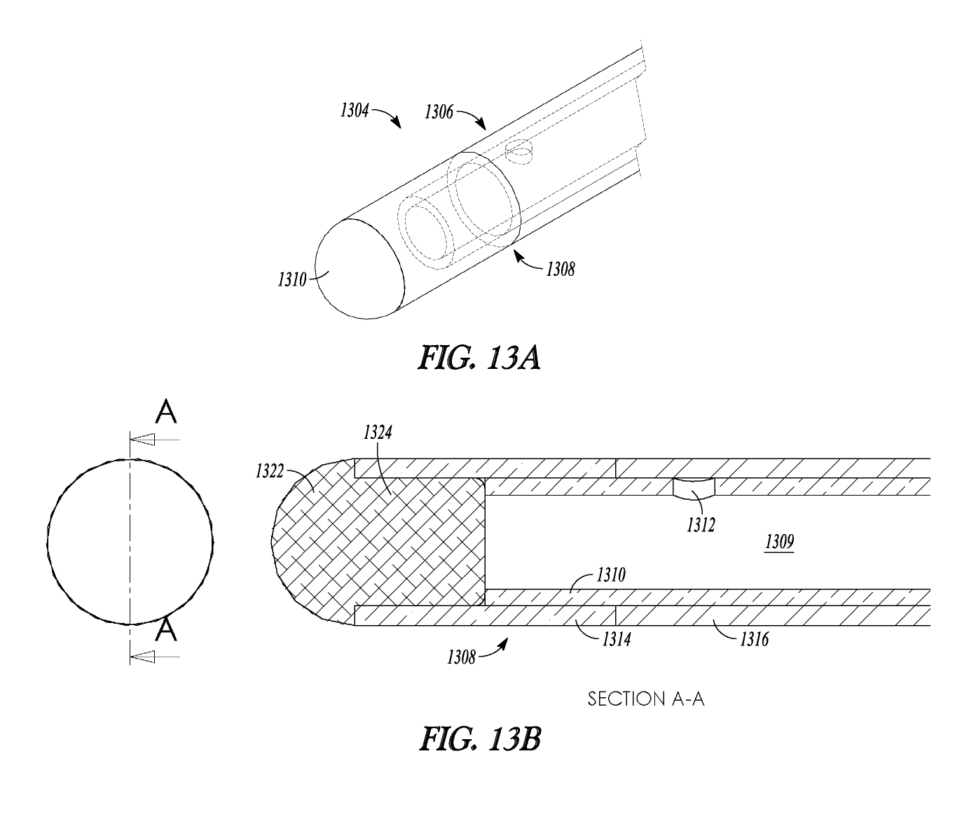

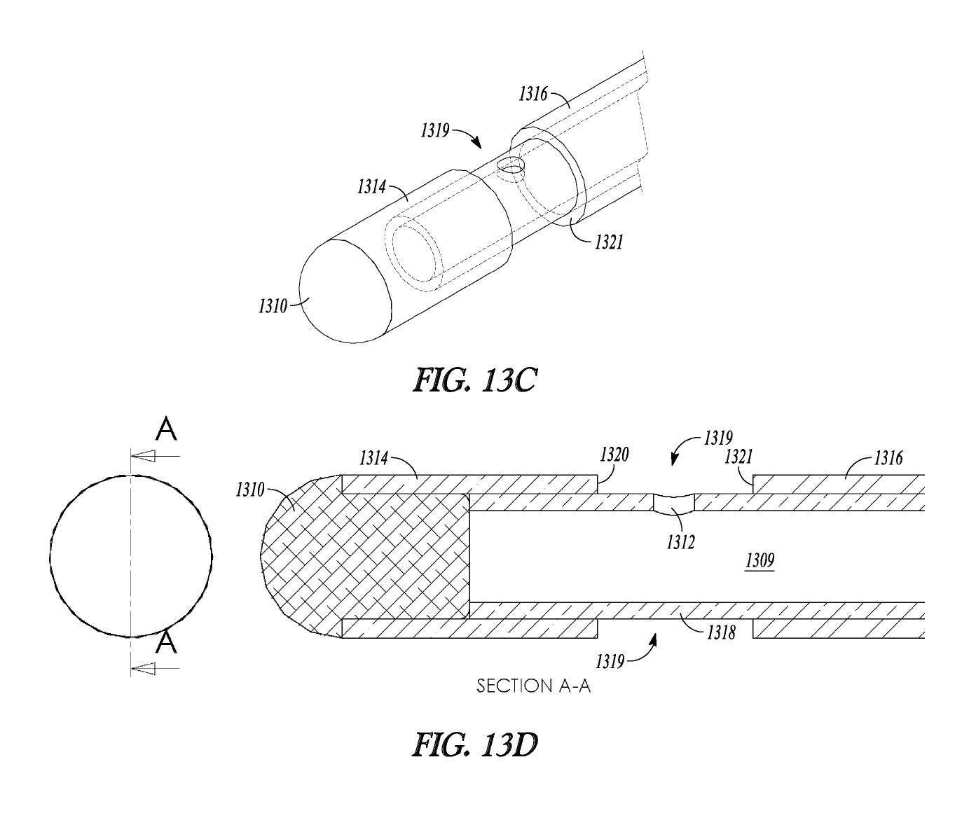

FIGS. 13A through 13D show an example of a distal portion 1316 of a catheter 1304, such as can be provided by the inner second catheter 104. The opening 1312 can be configured to be opened and closed, such as shown in the example of FIG. 13. The catheter 1304 can include a catheter body 1308 and a distal tip 1310. The distal portion 1306 of catheter body 1308 can include an outer shaft that can include a distal portion 1314 and a proximal portion 1316. The catheter body 1308 can include an inner shaft 1318. A bore 1309 can extend inside the inner shaft 1318, such as in the longitudinal direction toward a proximal portion of the catheter 1308. The inner shaft 1318 can include an opening 1312, which can be configured such as described above with respect to FIGS. 11 and 12 or elsewhere in this document.

In a closed configuration, such as shown in FIGS. 13A and 13B, the opening 1312 can be covered by the outer shaft, such as by the proximal outer shaft portion 1316. The distal outer shaft portion 1314 can be fixed to the inner shaft 1318, such as to permit the proximal outer shaft portion 1316 to slidably engage the inner shaft 1318.

In an open configuration, such as shown in FIGS. 13C and 13D, the proximal outer shaft portion 1316 can be retracted and an intermediate portion 1319 of the inner shaft 1318 providing the opening 1312 can be uncovered. The intermediate portion 1319 can form a recessed circumferential band around the catheter body 1308. The length or other dimensions of the intermediate portion 1319 can be similar to the recessed portion 1114 of FIGS. 11A and 11B. In an example, the proximal outer shaft portion 1316 can be stationary, and the inner shaft 1318 can be moved forward. To assist in the sliding motion, the proximal outer shaft portion or the inner shaft can include a biocompatible coating or lubricant, such as can be located between the moving portions of the components.

The edges 1320 and 1321 can be shaped to correspond to each other. Such corresponding shapes, which are shown as two vertical edges in the example shown in FIGS. 13A through 13D, can inhibit or avoid skiving of nearby or adjacent tissue by retracting the intermediate portion 1319, which can also inhibit or prevent infusate from leaking out of the opening 1312 until the intermediate portion 1319 is extended and the opening 1312 is exposed. Even if the edges 1320, 1321 are vertically oriented, they may be curved, tapered, beveled, chamfered, multifaceted, or angled. In an example, the edges 1320, 1321 can be shaped to provide corresponding mating inclined planes. To inhibit or prevent tissue skiving, when the proximal outer shaft portion 1316 is moved in the distal direction to close the opening 1312, the edge 1321 can be shaped to form an undercut or an inverse inclined plane. The edge 1320 would then be shaped as a mating inclined plane.

The distal tip 1310 can be comparable to or combined with one or more features of the distal tips 1110, 1210. Further details regarding examples of shapes, sizes, materials of the catheter body, the opening, the thin-walled portion or the distal tip are described in connection with FIGS. 11A and 11B, and in connection with FIGS. 12A-12B.

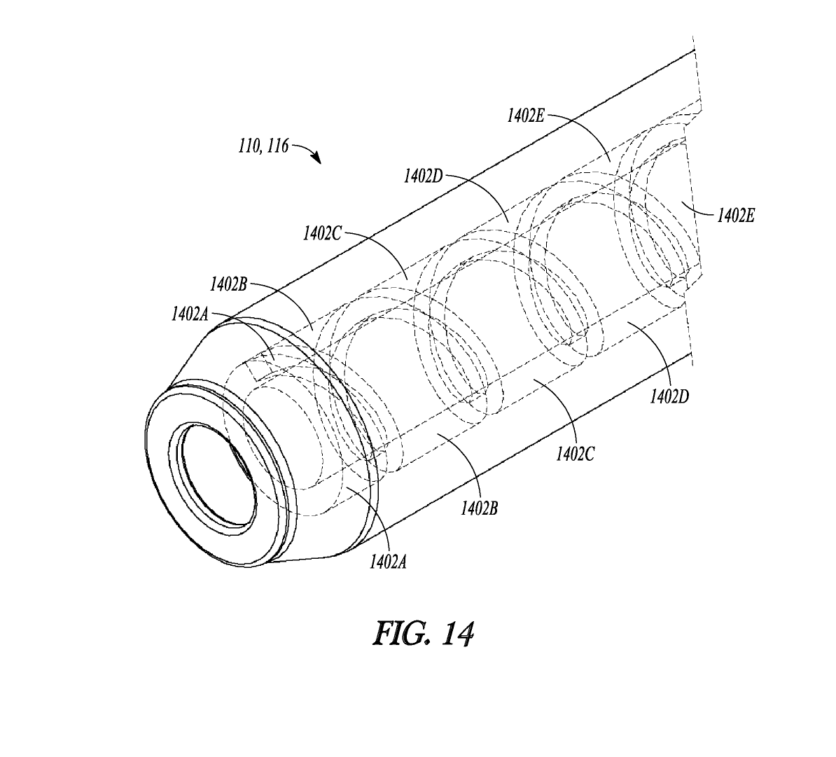

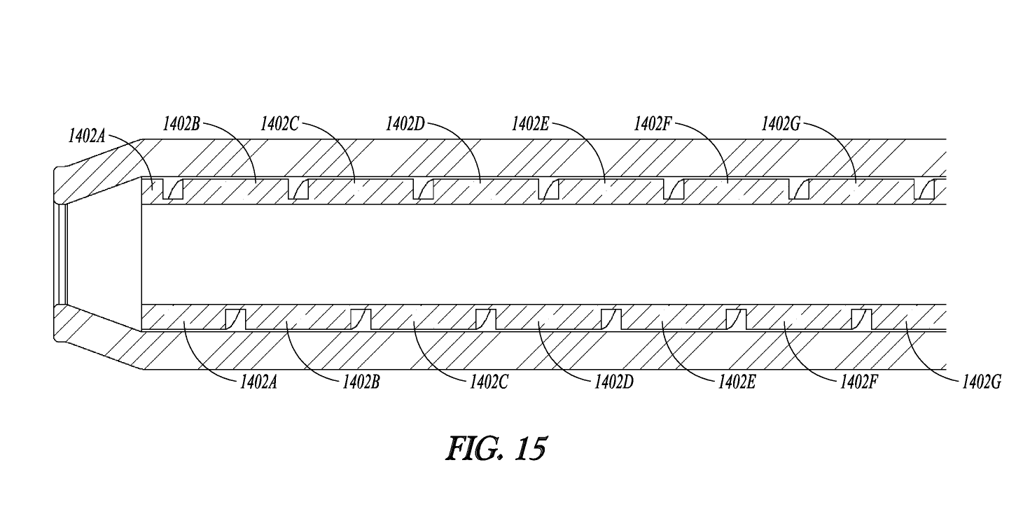

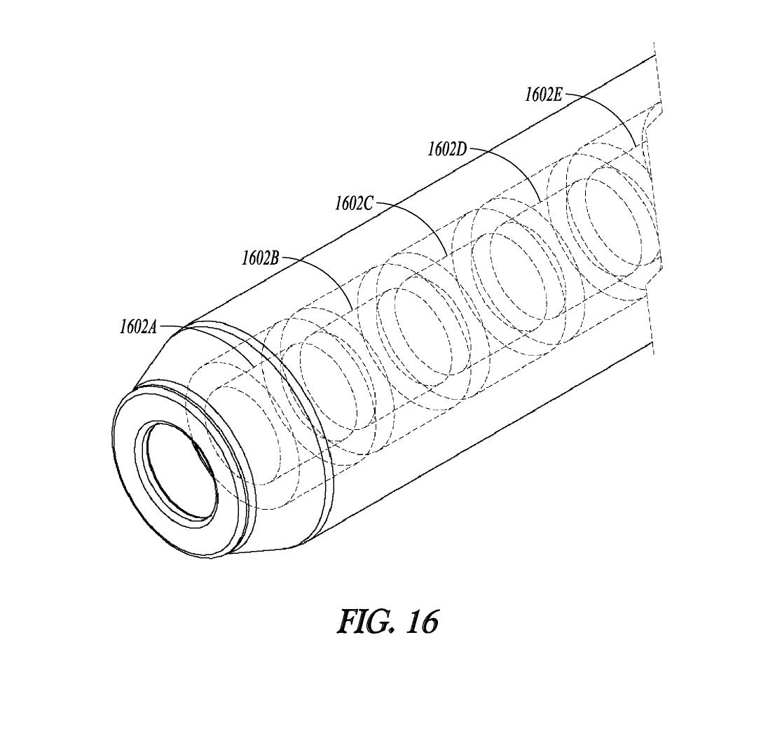

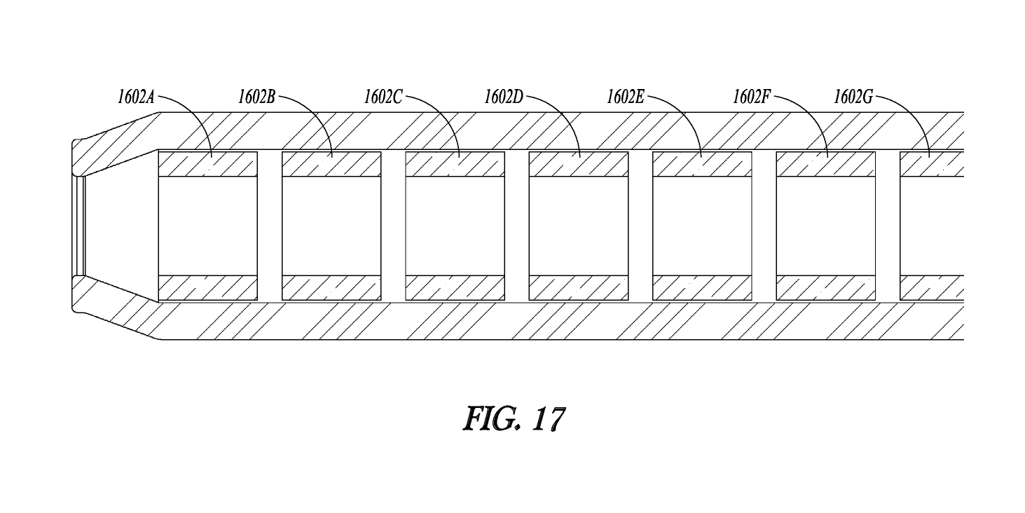

FIGS. 14-17 show an example of how at least one of the outer first catheter 102 body or the inner second catheter 104 body can be constructed so as to include different first and second materials providing different respective first and second stiffnesses, such as to provide a desired degree of flexibility, to provide a desired degree of kink resistance, or to provide a desired degree of flexibility and kink resistance. Although FIGS. 14-18 emphasize a distal portion 110, 116 of at least one of the catheters 102, 104, such as to more clearly illustrate construction details, it is understood that such construction can similarly be extended partially or all the way to the proximal portion 108, 114 of at least one of the catheters 102, 104.

In an example, the second material (e.g., polyimide or polyether ether ketone (PEEK)) can be stiffer than the first material (e.g., silicone or polyurethane). In an example, this can include using a braided or woven second material. In an additional or alternative example, the second material can be segmented, in a longitudinal direction, into a series of separate segments 1602A . . . 1602G or interconnected segments 1402A . . . 1402G. In an example, the interconnected segments 1402A . . . 1402G can use segmentation that can be provided at least in part by a spring (e.g., round wire, rectangular wire, or other spring) or spiral-cut second material, providing a spiral cut or groove between at least two adjacent segments 1402 in the longitudinal direction. The spiral cut can extend completely through the second material. The spiral groove can extend partially through the second material. In an example, the second material can be embedded in or otherwise joined by the softer first material, such as by using an overmolding, overextrusion, or shrinking process. In an example, the interconnected segments 1602A . . . 1602G can use segmentation that can be provided at least in part by at least two cylindrically separate adjacent segments 1602 that can be embedded in or otherwise joined by the first material, such as by using an overmolding process. The segments 1602 can be placed upon a mandrel and spaced apart at a desired separation distance by a spacer, which can then be removed, before the segments 1602 are embedded in or otherwise joined by the first material, such as by using the overmolding process.

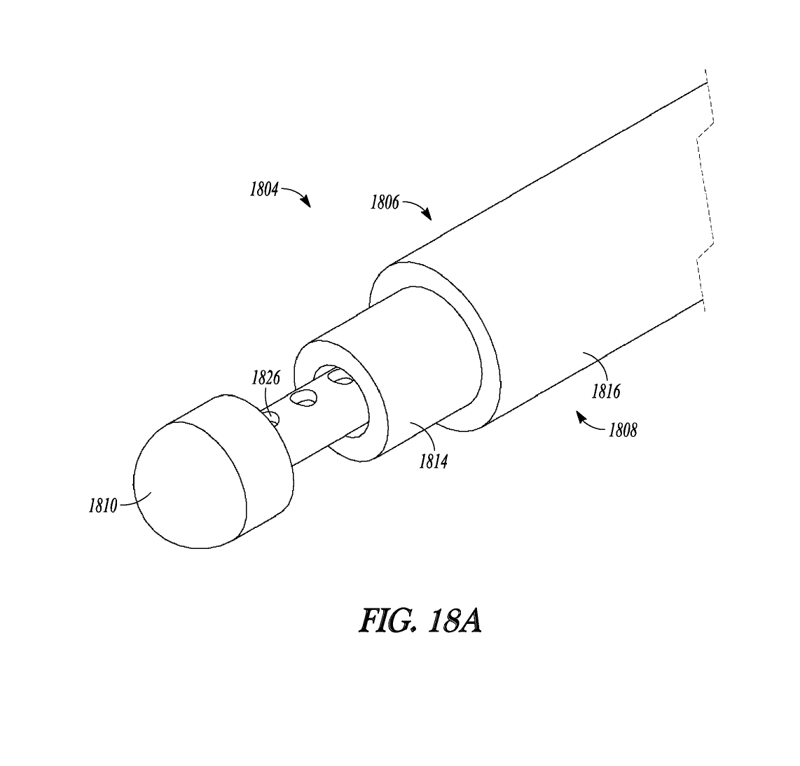

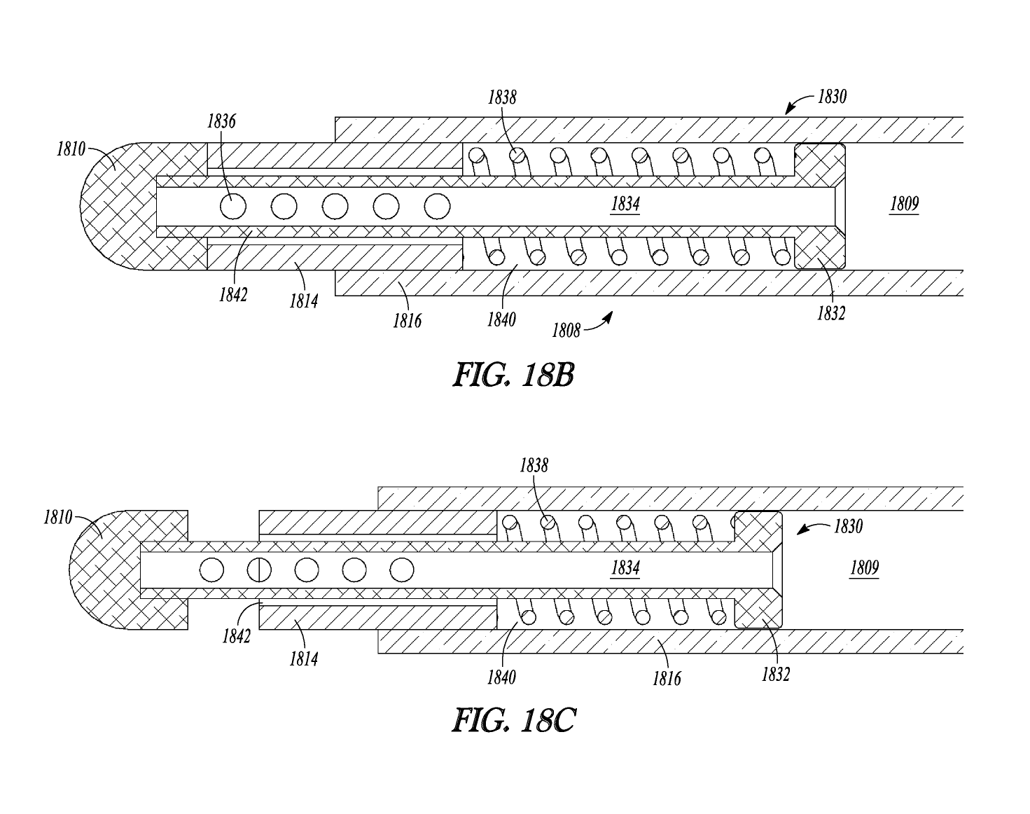

FIGS. 18A through 18C show an example of a distal portion 1806 of a second catheter 1804, such as can be provided by the inner second catheter 104. The second catheter 1804 can include a catheter body 1808. The catheter body 1808 can include an outer shaft 1816 and a distal inner shaft 1814. The distal inner shaft 1814 can be attached to the distal end of the outer shaft 1816, such as to project distally out of the outer shaft 1816. Both the inner shaft 1814 and the outer shaft 1816 can include a bore 1809, such as can extend between the proximal portion (not shown in the FIGS. 18A-18C) and the distal portion 1806. A plunger or piston 1830 can be situated inside the bore 1809. A base 1832 of the piston 1830 can face proximally such as toward the bore 1809. A bore 1834 of the piston 1830 can extend within a shaft from the base 1832 of the piston 1830 toward the distal tip of the piston 1830. The distal tip of the piston 1830 can be attached to the distal tip 1810 of the second catheter 1804. The bore 1834 of the piston 1830 can be in fluid communication with the bore 1809 of the outer shaft 1816.

FIGS. 18A and 18C show an example of an open configuration and FIG. 18B shows an example of a closed configuration of the second catheter 1804. In the closed configuration of FIG. 18B, the distal tip 1810 and the inner shaft 1814 can be in contact with each other, thereby closing a fluid passage between (1) bores 1809 and 1834 and (2) the tissue outside the outer periphery of the second catheter 1804. In the closed configuration, an infusate can fill the bores 1809 and 1834. In an example, an opening 1836 (e.g., along with further shown, but optional openings) can be sized or shaped or otherwise configured such that infusate only seeps in small or negligible quantities into a cavity 1840 through any clearance passageway 1842. In an example, the passageway 1842 (if any) can be sized such that capillary forces arising between the inner wall of the inner shaft 1814 and the outer wall of the bore shaft of the piston 1830 and the opening 1836 are such that such seeping into the cavity 1840 only occurs after a fluid pressure threshold P_max has been reached, which can provide a degree of pressure-relief. Alternatively or additionally, an O-ring or other seal can be included, such as can be inserted between a distal end of the spring 1838 and the proximal end of the inner shaft 1814, such as to inhibit or prevent fluid leakage into the cavity 1840.

In an example, when an infusate fills the bores 1809 and 1834, the fluid pressure inside the bores 1809 and 1834 rises. At a fluid pressure threshold P_open, where P_open<P_max, a spring or other resilient member, such as can be located in between the base 1832 of the piston 1830 and the proximal end of the distal portion of the inner shaft 1814 (or attached to the outer shaft 1816 or the distal tip 1810 in other examples) can be compressed due to the fluid pressure exerting P_open. In response, the piston 1830 can move in the distal direction. This can convert the closed configuration shown in the example of FIG. 18B to the open configuration shown in the examples of FIGS. 18A and 18C. When the piston 1830 is moved in the distal direction, a gap between the proximal end of the distal tip 1810 and the distal end of the inner shaft 1814 opens. This, in turn, can open up a fluid passage between (1) bores 1809, 1834 and (2) the surrounding tissue. Fluid can leave the bores through the opening 1836 and through any exposed adjacent openings. The number or area of exposed openings can serve to regulate the fluid pressure at which the infusate is delivered. The piston 1830 in connection with the resilient member (e.g., spring 1838) is only an example of a fluid-actuated pressure valve. Further examples can include one or any combination of a micro electrical-mechanical system, the use of a toroidal spring instead of a helical coil spring, or a slit or a membrane-covered opening, where the membrane is configured to withhold fluid, such as up to a certain predefined pressure, above which fluid can pass through the membrane or slit or other valve.

When the piston 1830 moves in the distal direction, the spring 1838 is compressed, which can allow fluid to exit the bores 1809, 1834 such as through the opening 1836, the pressure in the bores 1809, 1834 can drop below P_open after a short while and the resilient force of the resilient member can then push the piston 1830 back in the proximal direction, thereby closing the gap between the distal tip 1810 and the inner shaft 1814. Once the gap is closed and the catheter 1804 is in the closed configuration, the fluid left in the bores 1809, 1834 and further fluid supplied from the proximal portion of the second catheter can cause the pressure in the bores 1809, 1834 to rise again.

As the pressure of the fluid actuating the valve in the example of FIGS. 18A, 18B, and 18C is set to P_open, fluid exiting the opening 1836 does not leave the opening at pressures higher than P_open, if the mechanism to provide the fluid in the bore 1809 is a constant flow rate mechanism, such as is provided by many infusion pumps. The valved catheter in the example of FIGS. 18A, 18B, and 18C can be used as a pressure regulating mechanism, and can also be configured to provide a set amount of infusate to the region around the distal portion of the catheter, such as when the flow rate of the infusate entering the bores 1809, 1834 is kept constant. The dispensed amount or flow rate of infusate can be regulated, such as by changing the resilience of the valve, membrane, spring or MEMS to the desired setting. In an example, such changing can be user-provided, such as to allow user-adjustment of the dispensed amount or flow rate of infusate. For example, the user can be allowed to select or adjust the length of the inner shaft 1814, such as to change the displacement of the spring 1838, such as to pre-load the biasing force provided by the spring 1838 to a user-adjustable value. User selection or adjustment of any other valve characteristic can similarly be provided. These or other techniques for adjusting the biasing force provided by the spring 1838 could similarly be provided to the spring 1012 shown in the example of FIG. 10.

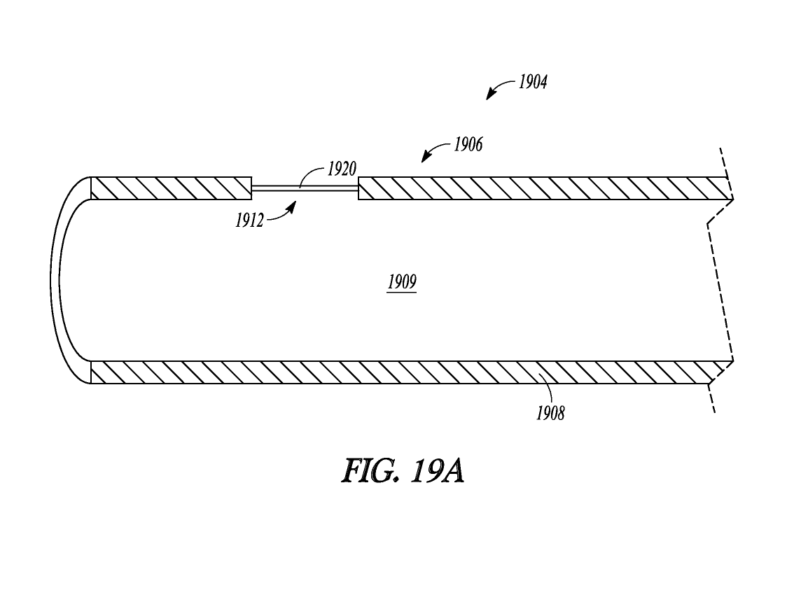

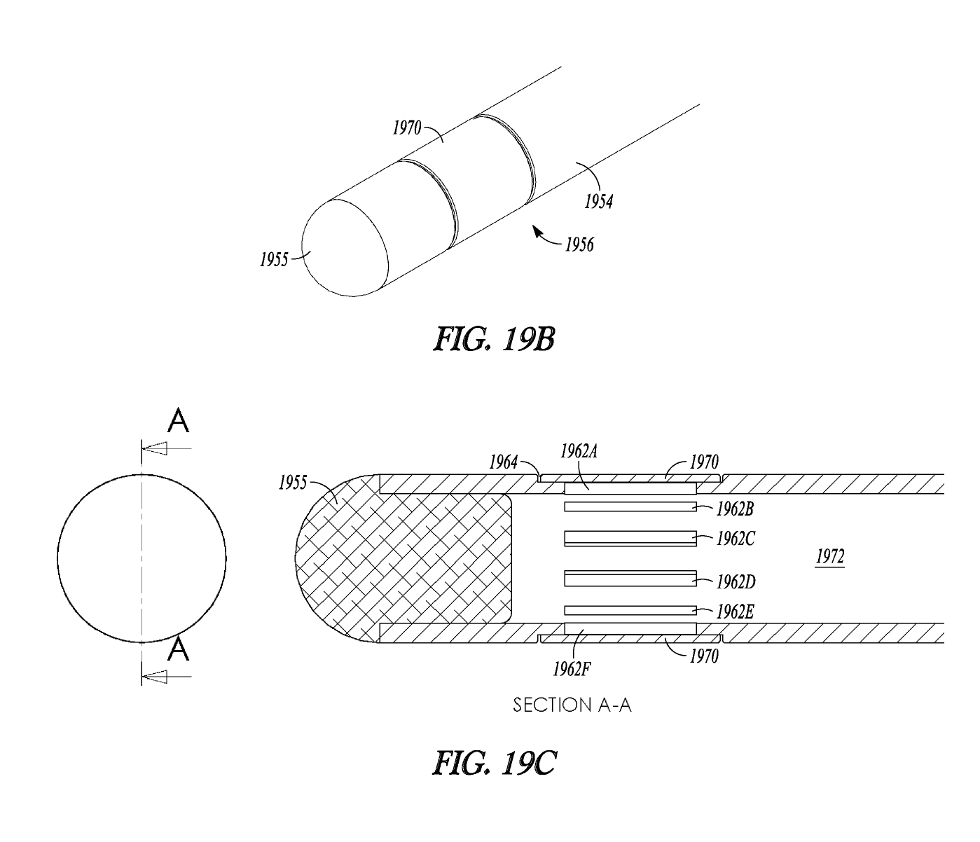

FIG. 19A shows an example of a distal portion 1906 of a second catheter 1904, such as can be provided by the inner second catheter 104. In an example, the distal portion 1906 of the second catheter 1904 can include at least one opening 1912. The opening 1912 can be covered by a slitted membrane 1920 or other covering that can include one or more slits. The at least one slit in the membrane 1920 can be configured to allow the membrane to open at a specified fluid pressure, and thereby provide a fluid passage between the bore 1909 and the tissue near the outer periphery of the second catheter 1904. In an example, the distal tip 1910 can be integrally connected with and made from the same material as the outer catheter body 1908. Further details regarding the catheter and possible modifications are found in the description of FIGS. 11-13 and 18 and in the other examples described in this document. The examples described with respect to FIGS. 18 and 19 in connection with an outer first catheter can be used to infuse about a distal portion of the outer first catheter when the opening 1836 is situated inside or outside the outer first catheter.

FIGS. 19B and 19C show an example of a distal portion 1956 of a second catheter 1954, such as can be provided by the inner second catheter 104. In an example, the distal portion 1956 of the second catheter 1954 can include one or more slitted, rounded, or other openings 1962, such as the openings 1962A-F shown in FIG. 19C. The openings 1962 can be located in a thin-walled or other recessed portion 1964 of the circumferential outer periphery of the distal portion 1956 of the second catheter 1954. The recessed portion 1964 can use details of examples described elsewhere in this document, such as with respect to FIG. 11A, 11B, 13C, or 13D. The recessed portion 1964 can be covered by a covering, such as a band 1970.

The band 1970 can be elastic. A fluid pressure within the bore 1972 of the catheter 1954 can be communicated via the openings 1962, such as to impart a force on the band 1970. This can stretch the band 1970 enough to allow delivery of the liquid infusate around the edges of the band 1970 to the nearby tissue. Thus, the band 1970 can serve to control or regulate a pressure or flow rate or other characteristic of delivering the liquid infusate to the subject.

Additionally or alternatively, the band 1970 can include a permeable material, such as a membrane. In an example, the band 1970 can be fluid permeable over at least a portion of the band. This can be used to control a pressure, flow rate, or other delivery characteristic of the liquid infusate from the bore 1972, through the openings 1962, through the permeable membrane, and to the tissue or other target region of the subject. In an example, the band 1970 can be selectively permeable, such as a selectively permeable membrane or other structure that can be configured to permit, control, or regulate a characteristic of delivery of a drug, a biologic, or other agent carried within the liquid of the infusate. In an example, the band 1970 can be selectively permeable to allow delivery of the agent while inhibiting or preventing the liquid of the infusate from passing through the band 1970.

A distal tip 1955 of the distal portion 1956 of the second catheter 1954 can be formed by filling the distal end portion of the bore 1972 of the distal portion 1956 of the second catheter 1954 with a material such as epoxy. Surface tension can be used to promote curing of the epoxy into a semispherical rounded distal tip 1955. Additionally or alternatively, the cured epoxy can be polished to smoothen it into a desired shape, such as the semispherical rounded distal tip 1955. The epoxy can be pre-loaded with a material to enhance its visibility on one or more imaging modalities, such as X-ray imaging, computed tomography (CT) imaging, magnetic resonance imaging (MR or MRI), ultrasound, or the like. Illustrative examples can include barium sulfate (BaSO₄), tungsten (W), tantalum (Ta), gold (Au), or any Noble metal or other material that is radiopaque such that it can enhance visibility of the distal tip 1955 of the catheter during X-ray, CT, or other radiographic imaging technique. Other illustrative examples include gadolinium (Gd), gadodiamide, or other paramagnetic, superparamagnetic or other material that can enhance visibility during MR imaging. The one or more imaging-enhancing materials can be provided as a powder that can be mixed into the epoxy, or as a liquid, such as a liquid capsule that can be embedded into the epoxy at or near the distal tip 1955. Using a combination of materials can allow multi-modal enhanced visibility using different imaging modalities. Although these details have been particularly described with respect to the distal tip 1955 shown in FIGS. 19B and 19C, they can also be applied to other catheter or stylet distal tip constructions described elsewhere in this document, such as the seal 124, the stylets 105, 106, the outer first catheter 102 or the inner second catheter 104, the distal tip 1122, 1124 of FIG. 11B, the distal tip 1210 of FIG. 12B, the distal tip 1322, 1324 of FIG. 13B, the distal tip 1310 of FIG. 13D, the distal tip 1810 of FIGS. 18A and 18B or other stylet or tip constructions discussed later in this document. Moreover, such details need not be limited to the distal tip, but can be applied to more proximal portions of one or more instruments, if enhanced imaging visibility of more than the distal tip is desired.

FIGS. 20-23 disclose examples of systems of a second catheter and a stylet to be inserted into a bore of the second catheter. Such systems can be configured to allow insertion and retraction of the stylet respectively into or from tissue beyond the distal end of the second catheter. When the stylet is pushed outside of the distal end of the second catheter, it can create a narrow gap in the tissue, such as can be observed, for example, in an MRI image after the stylet has been retracted into the bore of the second catheter.

The stylet of the different examples can include a tapered, beveled, chamfered or similar section that can narrow the lateral dimension of the stylet in a distal direction. Neighboring (e.g., more proximal) directions of the tapered section can be of a substantially constant cross-section, or can include one or more additional tapered or other variable-width cross-sections. A distal section of the stylet can provide more flexibility, such as due to its reduced cross-section. This can allow for improved insertional or navigational capabilities of the stylet, such as in comparison to a stylet without the tapered narrowed distal section. Inside the bore of the second catheter, the friction between the reduced cross-sectional area distal section of the stylet and the inner periphery of the second catheter can be reduced. Therefore, less force may be needed to push the stylet forward, such as during insertion into or beyond the catheter.

The distal section of the stylet can be pre-bent or can provide a pre-defined geometry. In an example, the distal section of the stylet can be constricted to conform to the geometry of the bore of the second catheter when the distal section of the stylet is inside the bore. When the distal end of the distal section exits the distal end of the second catheter, the distal section of the stylet can assert its pre-bent shape. In an example, one or more portions of the stylet can include one or more shape-memory materials (e.g., nitinol). In an example, the shape-memory material can be selected such that the shape-memory portion of the stylet can assert its pre-defined shape when exposed to the temperature of the surroundings, such as the body temperature of nearby tissue or body fluid. In an example, the distal section can switch into the pre-bent configuration when the surrounding temperature is slightly below human body temperature, for example, above 35° C.

While the distal section of the stylet can provide improved flexibility, the proximal section with its larger cross-section can give the stylet a higher rigidity, which can ease the insertion or guiding of the stylet through the bore of the second catheter. In order to position the proximal section of the stylet, the stylet can include a stop, which can be placed such that the stop can interact with the proximal end of the second catheter. The stop of the stylet can be user-adjustable in its position.

Alternatively, or additionally, the second catheter can optionally include a tapered section, which can optionally correspond to the tapered section of the stylet, for example, such as to act as a stop. The tapered section of the second catheter can be placed at the distal end of the second catheter.

When the stylet is placed inside the bore of the second catheter and the distal section of the stylet lies outside the distal end of the second catheter, the outer periphery of the distal section of the stylet can be used to allow infusate to travel from the bore to the distal tip of the stylet or along a tissue tract previously formed by the stylet. In examples of the system in which the stylet can be configured to transport infusate along its outer periphery, tissue nearby or surrounding the distal section can be contacted with infusate such as to increase the efficiency of the delivery of infusate to tissue. In an example, the stylet can be retracted before infusate is delivered into the tract in the tissue created by the stylet before its retraction and left behind after its retraction. In an example, after the infusate has been delivered, the stylet can be retracted, while the second catheter can remain in place in the tissue such as for later delivery of further infusate. In an example, the second catheter can also be retracted such that only the first catheter, through which the second catheter is introduced and removed, remains in the patient. In an example, the first catheter can also be removed after delivery of the infusate.

Between the stylet and the inner periphery of the second catheter, there can be a gap that can be shaped as an annular cross-intersection between the chosen geometry of the stylet and bore. In an example, both the stylet and the bore can have cross-sections of matching shape, for example, circular cross-sections; the gap or cross-intersection can then be an annular ring. In an example, the stylet can include a tapered section such that the area of the annular ring can depend on the position of the stylet within the bore. The annular ring defined by the distal section of the stylet and a fixed position along the inner periphery of the bore (proximal of a potentially present tapered section of the second catheter) can be larger than the annular ring defined by the proximal section of the stylet and the fixed position in the bore. In an example in which the second catheter includes a tapered section, the tapered section can be sized and shaped such that the annular ring between the tapered section of the second catheter and the tapered section of the stylet can be smaller, equal to, or larger than the annular ring defined by the proximal section of the stylet and the fixed position in the bore. The different relations between the annular shapes between the distal section of the stylet and the distal end of the second catheter, the tapered section of the stylet and the tapered section of the second catheter and the proximal section of the stylet and the fixed position in the second catheter can, in some examples, be used to regulate the amount and flux rate of delivery of infusate pumped into the bore of the second catheter from the catheter's proximal section.

It should be noted, however, that the interaction of the tapered section of the stylet and the tapered section of the second catheter (when the proximal section of the stylet is bigger the smallest cross-section of the tapered section of the second catheter) can be used to close the fluid passage between the bore and the surrounding tissue, such as by pushing the stylet further forward.

FIG. 20 shows a system 2000 comprising a second catheter 2004, such as can be provided by the inner second stylet 104, and a stylet 2006 that can be inserted into a bore 2009 of the second catheter 2004. The second catheter can include a catheter body 2008 and a tapered section 2010. In an example, the catheter body can be made from a rigid material that is rigid enough to withstand a lateral biasing applied by the shape-memory property of a portion of the stylet 2006 when the stylet 2006 is retracted into the bore 2009 of the second catheter 2004. Some illustrative examples of a suitably rigid material can include stainless steel, carbon fiber, ceramic, titanium. In an example, the tapered section 2010 can be made from a similarly rigid material, such as one of the above-listed example materials, or polyimide. The catheter body 2008 and the tapered section 2010 can be connected to each other, such as by welding or gluing using an adhesive, or can be integrally formed such as from the same material. The tapered section 2010 can include a proximal portion 2012 and a distal portion 2014.

The stylet 2006 can include a proximal section 2016, a tapered section 2018, and a distal section 2020. The distal section 2020 can include a stop 2022 which can be positioned such that the stylet 2006 cannot be moved further in the distal direction, such as shown in FIG. 20. The position of the stop 2022 can be chosen or adjusted by the manufacturer or by the end-user, such as to establish, maintain, or control a small gap between the tapered sections 2018 and 2012, such as to allow infusate to travel along the bore 2009 and along the outer periphery of distal section 2020 of the bore 2009.

In the configuration shown in FIG. 20, three annular rings can be defined. The first occurs between the distal section 2020 and the distal portion 2014 (denoted as area A), the second occurs between the tapered sections 2012 and 2018 (denoted as area B), and the third occurs between the proximal section 2016 and the outer periphery of the bore 2009 (denoted as area C) lying proximally of the tapered sections. The stylet 2006 and second catheter 2004 can be configured such that area C is slightly larger than area A. This can result in an infusate being ejected at the distal end of the tapered section 2014 at a slightly higher velocity than it is being inserted at the proximal portion of the second catheter 2009, due to the constriction at area A.

FIG. 21 shows an example of a system 2100 that can include a second catheter 2104, such as can be provided by the inner second catheter 104, and a stylet 2106. The second catheter 2104 can be geometrically identical to the catheter 2004. The stylet 2106 has been retracted from the position shown in FIG. 20.

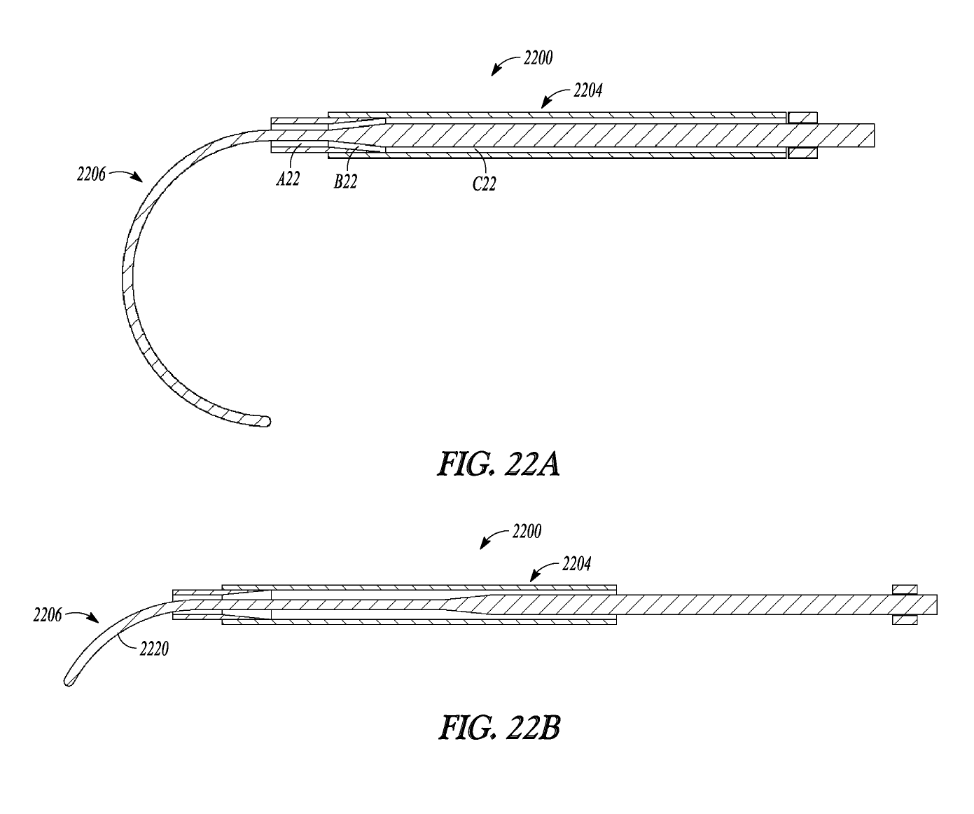

FIGS. 22A and 22B show an example of a different combination of a catheter 2204, such as can be provided by the inner second catheter 104, and a stylet 2206. For ease of explanation, the geometry of the second catheter 2204 can resemble the geometry of the catheters of FIGS. 20 and 21 again.

In FIG. 22A, examples of the areas A22, B22, and C22 are shown, all three areas respectively corresponding to areas A, B, and C of FIG. 20 in definition, albeit not in area itself. In an example such as shown in FIGS. 22A and 22B, the areas A22 and C22 can be equal in size, which can allow for a different flow profile of infusate than the flow profile of infusate of the example of FIG. 20. In FIG. 22B the distal section 2220 is shown in a state just before it is retracted inside the bore of distal end of the second catheter. As shown, the pre-bent (e.g., here specifically: pre-curved) geometry of the distal section is straightened out when pulled inside the bore.

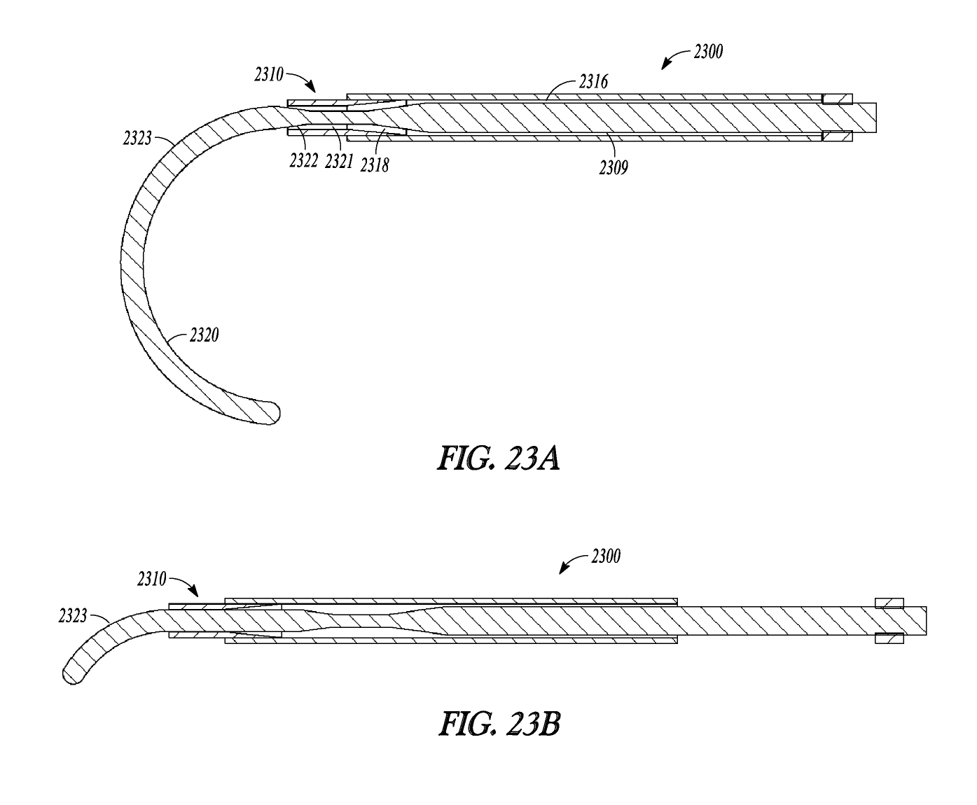

FIGS. 23A and 23B disclose another example of a system 2300 that can include a second catheter 2304, such as can be provided by the inner second catheter 104, and a tapered stylet 2306. In contrast to the stylet examples shown in FIGS. 20-22, the stylet 2306 can include a double-taper. A proximal portion 2316 of the stylet 2306 can end in a first tapered section 2318 which can end in distal section 2320, which can include an intermediate portion 2321, a tapered portion 2322, and an end portion 2323. In an example such as shown, the cross-sectional area of the end section 2323 can be substantially similar to the cross-sectional area of proximal section 2316, even though this relationship is only illustrative and may vary in some examples.

The end section 2323 can be used to substantially seal the distal end of the second catheter 2304 such as when the end section 2323 is positioned in the tapered section 2310 of the second catheter 2304. An example of position is shown in the example of FIG. 23B. By moving the stylet 2306 from the position shown in FIG. 23B to the position in FIG. 23A, the stylet 2306 can open a fluid passage between bore 2309 and the distal end of the second catheter 2304.

### Various Notes & Examples

Example 1 can include subject matter (such as an apparatus, a method, a means for performing acts, or a machine-readable medium including instructions that, when performed by the machine, cause the machine to perform acts) that can include or use a medical apparatus. The medical apparatus can include a first catheter. The first catheter can include a first catheter body that can include a proximal portion, a distal portion, and a longitudinal bore extending between the proximal portion of the first catheter body and the distal portion of the first catheter body, the longitudinal bore defining an inner circumferential periphery. The first catheter can include an elastically deformable first seal, located at the distal portion of the first catheter body. The first seal can be more flexible than a more proximal portion of the first catheter body. The first seal can define an inner circumferential periphery that, in a first configuration, can be laterally smaller than the inner circumferential periphery of a more proximal portion of the bore to at least partially occlude the bore at the distal portion of the catheter body and, in a second configuration, the inner circumferential periphery of the first seal can be expandable relative to the first configuration.

Example 2 can include, or can optionally be combined with the subject matter of Example 1 to include a second catheter, comprising a second catheter body including proximal portion, a distal portion, and a longitudinal bore extending between the proximal portion of the second catheter body and the distal portion of the second catheter body, wherein at least a portion of the second catheter body is sized and shaped to be inserted into or through and to slidingly engage the bore of the first catheter body, wherein the distal portion of the second catheter is laterally larger than the inner circumferential periphery of the first seal in its first configuration to expand the first seal into its second configuration. The first seal can be configured to elastically deform, in the second configuration, to conformally encompass an outer periphery of the second catheter when the second catheter has been inserted through the bore of the first catheter body such that the second catheter extends to an end or out of the distal portion of the first catheter body. The first seal at least partially occludes the bore at the end of the distal portion of the first catheter body in the first configuration in which there is an absence of the second catheter having been inserted through the bore of the first catheter body such that the second catheter extends to the end or out of the distal portion of the first catheter body.

Example 3 can include, or can optionally be combined with the subject matter of any one or more of Examples 1 or 2 to include the first seal is made from a different and more flexible material than a proximally adjacent portion of the first catheter body.

Example 4 can include, or can optionally be combined with the subject matter of any one or more of Examples 1 through 3 such that the first seal is integrally formed with a boot or jacket that encompasses an outer periphery of at least a portion of the first catheter body.

Example 5 can include, or can optionally be combined with the subject matter of any one or more of Examples 1 through 4 to include, in the second configuration, an outer dimension of the outer periphery of the first seal being laterally equal to or wider than a proximally adjacent outer dimension of the distal portion of the first catheter body.

Example 6 can include, or can optionally be combined with the subject matter of any one or more of Examples 1 through 5 to include wherein, in the second configuration, an outer dimension of the outer periphery of the first seal is laterally wider than a proximally adjacent outer dimension of the distal portion of the first catheter body by an amount that is sufficient to inhibit backflow of infusate outside of the first catheter body along a tract through tissue through which the first catheter body passes.

Example 7 can include, or can optionally be combined with the subject matter of any one or more of Examples 1 through 6 to include, in the second configuration, the first seal being sized and shaped to present a substantially laterally extending distal edge forming a substantially orthogonal or acute angle to a distal longitudinal direction of the first catheter body so as to inhibit backflow of infusate outside of the first catheter body along a tract through tissue through which the first catheter body passes.

Example 8 can include, or can optionally be combined with the subject matter of any one or more of Examples 1 through 7, to include a first stylet, having a distal portion that is laterally smaller than the inner circumferential periphery of the bore of the first catheter body, such that the first stylet is capable of being inserted into or through and to slidingly engage the bore of the first catheter body.

Example 9 can include, or can optionally be combined with the subject matter of any one or more of Examples 1 through 8 to include the distal portion of the first stylet is laterally larger than the inner circumferential periphery of the first seal in its first configuration to expand the first seal into its second configuration.

Example 10 can include, or can optionally be combined with the subject matter of any one or more of Examples 1 through 9 to include the first stylet comprising at least one of a rounded, tapered, or stepped-down distal tip.

Example 11 can include, or can optionally be combined with the subject matter of any one or more of Examples 1 through 10 to include a second catheter. The second catheter can include a second catheter body that can include a proximal portion, a distal portion, and a longitudinal bore extending between the proximal portion of the second catheter body and the distal portion of the second catheter body. At least a portion of the second catheter body can be sized and shaped to be inserted into or through and to slidingly engage the bore of the first catheter body. The distal portion of the second catheter can be laterally larger than the inner circumferential periphery of the first seal in its first configuration such as to expand the first seal into its second configuration. An elastically deformable second seal can be located at or toward the proximal portion of at least one of the first or second catheter body. The second seal can be configured to be elastically deformable to conformally encompass an outer periphery of a proximal portion of the other one of the first or second catheter body when at least a portion of the second catheter has been inserted into or through the bore of the first catheter body.

Example 12 can include, or can optionally be combined with the subject matter of any one or more of Examples 1 through 11 to include “the other” of the first or second catheter body including a seat protruding laterally outward from and peripherally about the proximal portion of the other of the first or second catheter, wherein the seat is sized and shaped to be positioned at least partially within and to be at least partially encompassed by the second seal.

Example 13 can include, or can optionally be combined with the subject matter of any one or more of Examples 1 through 12 to include, a second stylet, including at least a portion that is sized and shaped to be inserted into or through and to slidingly engage the bore of the second catheter body.

Example 14 can include, or can optionally be combined with the subject matter of any one or more of Examples 1 through 13 to include, a clamp or collar at the proximal portion of the second catheter, the clamp or collar configured to secure or seal the second stylet with respect to the second catheter body.

Example 15 can include, or can optionally be combined with the subject matter of any one or more of Examples 1 through 15 to include, a T-connector including a proximal port, a distal port opposing the proximal port, and a side port located between the proximal and distal ports, the T-connector configured such that the distal port of the T-connector is capable of being coupled to the proximal portion of the second catheter to allow infusion of a substance introduced via at least one of the proximal port or the side port.

Example 16 can include, or can optionally be combined with the subject matter of any one or more of Examples 1 through 15 to include, a second catheter. The second catheter can comprise a second catheter body including proximal portion, a distal portion, and a longitudinal bore extending between the proximal portion of the second catheter body and the distal portion of the second catheter body. At least a portion of the second catheter body can be sized and shaped to be inserted into or through and to slidingly engage the bore of the first catheter body. The distal portion of the second catheter can be laterally larger than the inner circumferential periphery of the first seal in its first configuration to expand the first seal into its second configuration. A second stylet can have a distal portion that is laterally smaller than the inner circumferential periphery of the bore of the second catheter body. The second stylet can be configured to be capable of being inserted into or through and to slidingly engage the bore of the second catheter body. The distal portion of the second stylet can include a shape-memory property such that the distal portion of the second stylet can curve away from a trajectory of the longitudinal bore of the second catheter body when the distal portion of the second stylet is extended beyond a distal end of the second catheter body. The apparatus can be configured such that the second catheter body can be longitudinally straight when the second stylet is retracted within the bore of the second catheter body.

Example 17 can include, or can optionally be combined with the subject matter of any one or more of Examples 1 through 16 to include a distal portion of the second catheter body, the bore of the second catheter tapers inward in a longitudinal direction away from the proximal portion of the second catheter body.

Example 18 can include, or can optionally be combined with the subject matter of any one or more of Examples 1 through 17 to include, a distal portion of the second stylet having a peripheral outer dimension that tapers inward in a longitudinal direction away from the proximal portion of the second stylet.

Example 19 can include, or can optionally be combined with the subject matter of any one or more of Examples 1 through 18 to include, the taper of the second stylet and the taper of the bore of the second catheter body are configured to seat against each other to seal against and thereby inhibit infusion of a substance via the bore of the second catheter body.

Example 20 can include, or can optionally be combined with the subject matter of any one or more of Examples 1 through 19 to include, the taper of the second stylet and the taper of the bore of the second catheter body being configured to be positioned with respect to each other to allow infusion of a substance via the bore of the second catheter body.

Example 21 can include, or can optionally be combined with the subject matter of any one or more of Examples 1 through 20 to include, a distal annular clearance opening, between the second stylet and the inner circumferential periphery of the bore of the second catheter body at the distal end of the second catheter body, is large enough to allow infusion with the second stylet extending out beyond the distal end of the second catheter body.

Example 22 can include, or can optionally be combined with the subject matter of one or more of Examples 1 through 21 to include, a second stylet that is tapered outward in or to a more proximal portion of the bore of the second catheter at which a more proximal annular clearance opening exists between the second stylet and the inner circumferential periphery of the bore of the second catheter body, and wherein a cross-sectional area of the distal annular clearance opening is equal or greater to a cross-sectional area of the more proximal annular clearance opening.

Example 23 can include, or can optionally be combined with the subject matter of one or more of Examples 1 through 22 to include, a distal annular clearance opening, between the second stylet and the inner circumferential periphery of the bore of the second catheter body at the distal end of the second catheter body, in a first configuration in which the shape-memory portion of the second stylet is retracted into the bore of the second catheter body, is small enough to inhibit or prevent infusion in the first configuration, but wherein the distal annular clearance opening in a second configuration in which the shape-memory portion of the second stylet is extended out from the bore of the second catheter body, is large enough to permit infusion in the second configuration.

Example 24 can include, or can optionally be combined with the subject matter of one or more of Examples 1 through 23 to include, a proximal portion of a second stylet that can be more rigid than a distal portion of the second stylet.

Example 25 can include, or can optionally be combined with the subject matter of one or more of Examples 1 through 24 to include, a proximal portion of a second stylet that can include a clamp or collar configured to limit insertion, into the bore of the second catheter body, of the portion of the second catheter body that is sized and shaped to be inserted into or through and to slidingly engage the bore of the first catheter body.

Example 26 can include, or can optionally be combined with the subject matter of one or more of Examples 1 through 25 to include, the clamp or collar being configured to be user-adjustable to permit a user to adjust an insertion limit provided by the clamp or collar.

Example 27 can include, or can optionally be combined with the subject matter of one or more of Examples 1 through 26 to include, a clamp or collar that can be configured to engage at least one of the first catheter body or the second catheter body.

Example 28 can include, or can optionally be combined with the subject matter of one or more of Examples 1 through 27 to include, a clamp or collar that can be configured to inhibit rotation of the second stylet with respect to at least one of the first catheter body or the second catheter body when the clamp or collar is engaged with at least one of the first catheter body or the second catheter body.

Example 29 can include, or can optionally be combined with the subject matter of one or more of Examples 1 through 28 to include, a clamp or collar that can be capable of being configured to limit insertion such that the spacing between the second stylet and the bore of the second catheter inhibits or prevents infusion of a fluid through a distal portion of the bore of the second catheter.

Example 30 can include, or can optionally be combined with the subject matter of one or more of Examples 1 through 29 to include, a clamp or collar that can be capable of being configured to limit insertion such that the spacing between the second stylet and the bore of the second catheter permits infusion of a fluid through a distal portion of the bore of the second catheter.

Example 31 can include, or can optionally be combined with the subject matter of one or more of Examples 1 through 30 to include, a clamp or collar that can be capable of being configured to limit insertion to allow user-selection between: a spacing between the second stylet and the bore of the second catheter inhibiting or preventing infusion of a fluid through a distal portion of the bore of the second catheter; and a spacing between the second stylet and the bore of the second catheter permitting infusion of a fluid through a distal portion of the bore of the second catheter.

Example 32 can include, or can optionally be combined with the subject matter of one or more of Examples 1 through 30 to include a pressure-relief valve assembly, which can be configured to be in fluid communication with the longitudinal bore of the second catheter. The pressure relief valve assembly can be configured to open in response to a fluid pressure within the longitudinal bore of the second catheter meeting or exceeding a specified maximum pressure threshold value.

Example 33 can include, or can optionally be combined with the subject matter of one or more of Examples 1 through 32 to include, a pressure relief valve assembly that comprises an input port, configured to be in fluid communication with the longitudinal bore of the second catheter; an output port or reservoir; and a valve, configured to seal the input port when a fluid pressure of a fluid that is in fluid communication with the longitudinal bore of the second catheter is less than the specified maximum pressure threshold value, and configured to vent fluid from the input port to the output port or reservoir in response to the fluid pressure meeting or exceeding the specified maximum pressure threshold value.

Example 34 can include, or can optionally be combined with the subject matter of one or more of Examples 1 through 33 to include a valve that includes a ball and a spring, wherein the spring biases the ball, and wherein the specified maximum pressure threshold value depends at least in part on a spring constant characteristic of the spring.

Example 35 can include, or can optionally be combined with the subject matter of one or more of Examples 1 through 34 to include, an output port or reservoir that comprises: an exit passage; and a membrane sealing the exit passage, the membrane configured to allow passage of air and to inhibit passage of liquid fluid.

Example 36 can include, or can optionally be combined with the subject matter of one or more of Examples 1 through 35 to include a T-connector or a Y-connector including a proximal port, a distal port opposing the proximal port, and a side port located between the proximal and distal ports, the T-connector or Y-connector configured such that the distal port is capable of being coupled to the proximal portion of the second catheter to allow infusion of a substance introduced at the proximal port or the side port. A pressure relief valve can be in fluid communication with the longitudinal bore of the second catheter via the side port of the T-connector or Y-connector.

Example 37 can include, or can optionally be combined with the subject matter of any one or more of Examples 1 through 36 to include, a valve assembly, located at the distal portion of the second catheter, the valve assembly configured to be in fluid communication with the longitudinal bore of the second catheter, the valve assembly including a plunger or other actuator configured to open an infusion port in response to a sensed fluid pressure in the bore of the second catheter meeting or exceeding a specified pressure threshold value.

Example 38 can include, or can optionally be combined with the subject matter of any one or more of Examples 1 through 37 to include, a plunger or other actuator comprises a biasing spring that is configured to control opening of a selected infusion port number or area to regulate fluid pressure in the bore of the second catheter.

Example 39 can include, or can optionally be combined with the subject matter of any one or more of Examples 1 through 38 to include, at least one of the first catheter body or the second catheter body including different first and second materials providing different respective first and second stiffnesses. The second material can be stiffer than the first material. The second material can be segmented into a series of separate or interconnected segments in a longitudinal direction.

Example 40 can include, or can optionally be combined with the subject matter of one or more of Examples 1 through 39 to include, segmentation that can be provided at least in part by a spiral-cut second material, providing a spiral cut or groove between at least two adjacent segments in the longitudinal direction.

Example 41 can include, or can optionally be combined with the subject matter of one or more of Examples 1 through 39 to include, segmentation segmentation is provided at least in part by at least two cylindrically separate adjacent segments joined by the first material.

Example 42 can include, or can optionally be combined with the subject matter of one or more of Examples 1 through 41 to include, a catheter body having a first material that comprises at least one of silicone or polyurethane and wherein the second material comprises at least one of polyimide or polyether ether ketone (PEEK).

Example 43 can include, or can optionally be combined with the subject matter of one or more of Examples 1 through 42 to include, a second catheter, comprising a second catheter body including a proximal portion, a distal portion, and a longitudinal bore extending in a longitudinal direction between the proximal portion of the second catheter body and the distal portion of the second catheter body, wherein at least a portion of the second catheter body is sized and shaped to be inserted into or through the bore of the first catheter body. The second catheter can include a distal tip and at least one opening in the second catheter body fluidly connecting the bore and an outer periphery of the second catheter.

Example 44 can include, or can optionally be combined with the subject matter of one or more of Examples 1 through 43 to include, an opening including a varying cross-sectional area in a direction substantially perpendicular to the longitudinal direction.

Example 45 can include, or can optionally be combined with the subject matter of one or more of Examples 1 through 44 to include, a second catheter that can comprises an intermediate portion comprising an indentation or groove about the opening.

Example 46 can include, or can optionally be combined with the subject matter of one or more of Examples 1 through 45 to include a second catheter body that comprises an outer shaft and an inner shaft, the inner shaft including the opening and at least a portion of the outer shaft slidably engaging the inner shaft.

Example 47 can include, or can optionally be combined with the subject matter of one or more of Examples 1 through 46 to include, a valve is located in the distal portion of the second catheter to open an opening connecting the bore and region outside the second catheter.

Example 48 can include, or can optionally be combined with the subject matter of any one or more of Examples 1 though 47 to include, a valve that comprises a piston slidably engaged in the bore, the piston comprising the at least one opening; the opening being covered by a portion of the catheter body in a closed configuration and the opening being open in an open configuration, and a resilient member positioned between the piston and the distal tip actuating the movement of the opening from the closed to the open configuration.

Example 49 can include, or can optionally be combined with the subject matter of any one or more of Examples 1 through 48 to include, a method for delivering a catheter system that can include a first catheter body including a proximal portion, a distal portion, and a longitudinal bore extending between the proximal portion of the first catheter body and the distal portion of the first catheter body, the longitudinal bore defining an inner circumferential periphery, and an elastically deformable first seal, located at the distal portion of the first catheter body, the first seal being more flexible than a more proximal portion of the first catheter body, the first seal defining an inner circumferential periphery that, in a first configuration, is laterally smaller than the inner circumferential periphery of a more proximal portion of the bore to at least partially occlude the bore at the distal portion of the catheter body and, in a second configuration, the inner circumferential periphery of the first seal is expandable relative to the first configuration. In the method, a stylet can be positioned inside the bore of the first catheter, when the first catheter is guided to a target region.

Example 50 can include, or can optionally be combined with the subject matter of any one or more of Examples 1 through 49 to include, inserting a second catheter inside the first catheter, the second catheter comprising a bore.

Example 51 can include, or can optionally be combined with the subject matter of any one or more of Examples 1 through 50 to include, removing the stylet from the bore of the first catheter before the second catheter is inserted.

Example 52 can include, or can optionally be combined with the subject matter of any one or more of Examples 1 through 51 to include, advancing the second catheter within the bore of the first catheter such that a distal tip of the second catheter extends from a most distal tip of the first seal to transition the first configuration of the first seal to its second configuration.

Example 53 can include, or can optionally be combined with the subject matter of any one or more of Examples 1 through 52 to include, the second catheter comprising an opening for supplying a fluid to the target region, and the step of positioning the second catheter within the bore of the first catheter such that the first seal conformally encompasses an outer periphery of the second catheter.

Example 54 can include, or can optionally be combined with the subject matter of any one or more of Examples 1 through 53 to include, the first seal conformally encompassing the outer periphery of the second catheter to inhibit or prevent reflux, and including the step of supplying fluid through a bore of the second catheter to exit at the opening and to deliver the fluid.

Example 55 can include, or can optionally be combined with the subject matter of any one or more of Examples 1 through 54 to include, the fluid being delivered to the target region depending on the pressure of the fluid in the bore of the second catheter.

Example 56 can include, or can optionally be combined with the subject matter of any one or more of Examples 1 through 55 to include the step of regulating a fluid flow within out of the bore of the second catheter by a stylet positioned in said bore.

Example 57 can include, or can optionally be combined with the subject matter of any one or more of Examples 1 through 56 to include, leaving the second catheter remaining in proximity of the target region between subsequent fluid deliveries to the target region.

Example 58 can include, or can optionally be combined with the subject matter of any one or more of Examples 1 through 57 to include, the first catheter remaining in proximity of the target region between subsequent fluid deliveries to the target region.

Example 59 can include, or can optionally be combined with the subject matter of any one or more of Examples 1 through 58 to include the stylet extending from a most distal tip of the first seal to prevent coring of tissue.

Each of these non-limiting examples can stand on its own, or can be combined with one or more of the other examples in any permutation or combination.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A medical apparatus comprising:
a first catheter, comprising:
a first catheter body including a proximal portion, a distal portion, and a longitudinal bore extending between the proximal portion of the first catheter body and the distal portion of the first catheter body, the longitudinal bore defining an inner circumferential periphery; and
an elastically deformable first seal, located at the distal portion of the first catheter body, the first seal being more flexible than a more proximal portion of the first catheter body, the first seal defining an inner circumferential periphery that, in a first configuration, is laterally smaller than the inner circumferential periphery of a more proximal portion of the bore to at least partially occlude the bore at the distal portion of the catheter body and, in a second configuration, the inner circumferential periphery of the first seal is expandable relative to the first configuration.

2. The apparatus of claim 1, comprising a second catheter, comprising a second catheter body including proximal portion, a distal portion, and a longitudinal bore extending between the proximal portion of the second catheter body and the distal portion of the second catheter body, wherein at least a portion of the second catheter body is sized and shaped to be inserted into or through and to slidingly engage the bore of the first catheter body, wherein the distal portion of the second catheter is laterally larger than the inner circumferential periphery of the first seal in its first configuration to expand the first seal into its second configuration; and
wherein the first seal is configured to elastically deform, in the second configuration, to conformally encompass an outer periphery of the second catheter when the second catheter has been inserted through the bore of the first catheter body such that the second catheter extends to an end or out of the distal portion of the first catheter body; and
wherein the first seal at least partially occludes the bore at the end of the distal portion of the first catheter body in the first configuration in which there is an absence of the second catheter having been inserted through the bore of the first catheter body such that the second catheter extends to the end or out of the distal portion of the first catheter body.

3. (canceled)

4. (canceled)

5. (canceled)

6. The apparatus of claim 1, wherein, in the second configuration, an outer dimension of the outer periphery of the first seal is laterally wider than a proximally adjacent outer dimension of the distal portion of the first catheter body by an amount that is sufficient to inhibit backflow of infusate outside of the first catheter body along a tract through tissue through which the first catheter body passes.

7. The apparatus of claim 1, wherein, in the second configuration, the first seal is sized and shaped to present a substantially laterally extending distal edge forming a substantially orthogonal or acute angle to a distal longitudinal direction of the first catheter body so as to inhibit backflow of infusate outside of the first catheter body along a tract through tissue through which the first catheter body passes.

8. The apparatus of claim 1, comprising a first stylet, having a distal portion that is laterally smaller than the inner circumferential periphery of the bore of the first catheter body, such that the first stylet is capable of being inserted into or through and to slidingly engage the bore of the first catheter body, wherein the distal portion of the first stylet is laterally larger than the inner circumferential periphery of the first seal in its first configuration to expand the first seal into its second configuration, wherein the first stylet comprises at least one of a rounded, tapered, or stepped-down distal tip.

9. (canceled)

10. (canceled)

11. The apparatus of claim 1, comprising:
a second catheter, comprising a second catheter body including proximal portion, a distal portion, and a longitudinal bore extending between the proximal portion of the second catheter body and the distal portion of the second catheter body, wherein at least a portion of the second catheter body is sized and shaped to be inserted into or through and to slidingly engage the bore of the first catheter body, wherein the distal portion of the second catheter is laterally larger than the inner circumferential periphery of the first seal in its first configuration to expand the first seal into its second configuration; and
an elastically deformable second seal, located at or toward the proximal portion of at least one of the first or second catheter body, the second seal configured to be elastically deformable to conformally encompass an outer periphery of a proximal portion of the other one of the first or second catheter body when at least a portion of the second catheter has been inserted into or through the bore of the first catheter body.

12. The apparatus of claim 11, wherein the other of the first or second catheter body includes a seat protruding laterally outward from and peripherally about the proximal portion of the other of the first or second catheter, wherein the seat is sized and shaped to be positioned at least partially within and to be at least partially encompassed by the second seal.

13. The apparatus of claim 11, comprising a second stylet, including at least a portion that is sized and shaped to be inserted into or through and to slidingly engage the bore of the second catheter body, and comprising a clamp or collar at the proximal portion of the second catheter, the clamp or collar configured to secure or seal the second stylet with respect to the second catheter body.

14. (canceled)

15. The apparatus of claim 11, comprising a T-connector including a proximal port, a distal port opposing the proximal port, and a side port located between the proximal and distal ports, the T-connector configured such that the distal port of the T-connector is capable of being coupled to the proximal portion of the second catheter to allow infusion of a substance introduced via at least one of the proximal port or the side port.

16. The apparatus of claim 10, comprising:
a second catheter, comprising a second catheter body including proximal portion, a distal portion, and a longitudinal bore extending between the proximal portion of the second catheter body and the distal portion of the second catheter body, wherein at least a portion of the second catheter body is sized and shaped to be inserted into or through and to slidingly engage the bore of the first catheter body, wherein the distal portion of the second catheter is laterally larger than the inner circumferential periphery of the first seal in its first configuration to expand the first seal into its second configuration;
a second stylet, having a distal portion that is laterally smaller than the inner circumferential periphery of the bore of the second catheter body, such that the second stylet is capable of being inserted into or through and to slidingly engage the bore of the second catheter body;
wherein the distal portion of the second stylet includes a shape-memory property such that the distal portion of the second stylet curves away from a trajectory of the longitudinal bore of the second catheter body when the distal portion of the second stylet is extended beyond a distal end of the second catheter body; and
wherein the apparatus is configured such that the second catheter body is longitudinally straight when the second stylet is retracted within the bore of the second catheter body.

17. The apparatus of claim 16, wherein, at a distal portion of the second catheter body, the bore of the second catheter tapers inward in a longitudinal direction away from the proximal portion of the second catheter body, wherein a distal portion of the second stylet has a peripheral outer dimension that tapers inward in a longitudinal direction away from the proximal portion of the second stylet.

18. (canceled)

19. (canceled)

20. (canceled)

21. (canceled)

22. (canceled)

23. The apparatus of claim 16, wherein a distal annular clearance opening, between the second stylet and the inner circumferential periphery of the bore of the second catheter body at the distal end of the second catheter body, in a first configuration in which the shape-memory portion of the second stylet is refracted into the bore of the second catheter body, is small enough to inhibit or prevent infusion in the first configuration, but wherein the distal annular clearance opening in a second configuration in which the shape-memory portion of the second stylet is extended out from the bore of the second catheter body, is large enough to permit infusion in the second configuration.

24. (canceled)

25. The apparatus of claim 16, wherein the proximal portion of the second stylet includes a clamp or collar configured to limit insertion, into the bore of the second catheter body, of the portion of the second catheter body that is sized and shaped to be inserted into or through and to slidingly engage the bore of the first catheter body, wherein the clamp or collar is configured to be user-adjustable to permit a user to adjust an insertion limit provided by the clamp or collar, wherein the clamp or collar is configured to engage at least one of the first catheter body or the second catheter body, wherein the clamp or collar is configured to inhibit rotation of the second stylet with respect to at least one of the first catheter body or the second catheter body when the clamp or collar is engaged with at least one of the first catheter body or the second catheter body.

26. (canceled)

27. (canceled)

28. (canceled)

29. (canceled)

30. (canceled)

31. The apparatus of claim 25, wherein the clamp or collar is capable of being configured to limit insertion to allow user-selection between:
a spacing between the second stylet and the bore of the second catheter inhibiting or preventing infusion of a fluid through a distal portion of the bore of the second catheter; and
a spacing between the second stylet and the bore of the second catheter permitting infusion of a fluid through a distal portion of the bore of the second catheter.

32. The apparatus of claim 2, comprising:
a pressure-relief valve assembly, configured to be in fluid communication with the longitudinal bore of the second catheter, the pressure relief valve assembly configured to open in response to a fluid pressure within the longitudinal bore of the second catheter meeting or exceeding a specified maximum pressure threshold value.

33. The apparatus of claim 32, wherein the pressure relief valve assembly comprises:
an input port, configured to be in fluid communication with the longitudinal bore of the second catheter;
an output port or reservoir, wherein the output port or reservoir comprises:
an exit passage; and
a membrane sealing the exit passage, the membrane configured to allow passage of air and to inhibit passage of liquid fluid; and
a valve, configured to seal the input port when a fluid pressure of a fluid that is in fluid communication with the longitudinal bore of the second catheter is less than the specified maximum pressure threshold value, and configured to vent fluid from the input port to the output port or reservoir in response to the fluid pressure meeting or exceeding the specified maximum pressure threshold value.

34. (canceled)

35. (canceled)

36. The apparatus of claim 32, comprising:
a T-connector or a Y-connector including a proximal port, a distal port opposing the proximal port, and a side port located between the proximal and distal ports, the T-connector or Y-connector configured such that the distal port is capable of being coupled to the proximal portion of the second catheter to allow infusion of a substance introduced at the proximal port or the side port; and
wherein the pressure relief valve is in fluid communication with the longitudinal bore of the second catheter via the side port of the T-connector or Y-connector.

37. The apparatus of claim 2, comprising:
a valve assembly, located at the distal portion of the second catheter, the valve assembly configured to be in fluid communication with the longitudinal bore of the second catheter, the valve assembly including a plunger or other actuator configured to open an infusion port in response to a sensed fluid pressure in the bore of the second catheter meeting or exceeding a specified pressure threshold value.

38. (canceled)

39. The apparatus of claim 1, wherein at least one of the first catheter body or the second catheter body includes different first and second materials providing different respective first and second stiffnesses, wherein the second material is stiffer than the first material, and wherein the second material is segmented into a series of separate or interconnected segments in a longitudinal direction.

40. (canceled)

41. (canceled)

42. (canceled)

43. The apparatus of claim 1, comprising:
a second catheter, comprising a second catheter body including a proximal portion, a distal portion, and a longitudinal bore extending in a longitudinal direction between the proximal portion of the second catheter body and the distal portion of the second catheter body, wherein at least a portion of the second catheter body is sized and shaped to be inserted into or through the bore of the first catheter body; and
the second catheter comprising a distal tip and at least one opening in the second catheter body fluidly connecting the bore and an outer periphery of the second catheter, the opening including a varying cross-sectional area in a direction substantially perpendicular to the longitudinal direction.

44. (canceled)

45. (canceled)

46. The apparatus of claim 43, wherein the second catheter body comprises an outer shaft and an inner shaft, the inner shaft including the opening and at least a portion of the outer shaft slidably engaging the inner shaft.

47. The apparatus of claim 43, wherein a valve is located in the distal portion of the second catheter to open an opening connecting the bore and region outside the second catheter, wherein the valve comprises a piston slidably engaged in the bore, the piston comprising the at least one opening; the opening being covered by a portion of the catheter body in a closed configuration and the opening being open in an open configuration, and a resilient member positioned between the piston and the distal tip actuating the movement of the opening from the closed to the open configuration.

48. (canceled)

49. The apparatus of claim, wherein, in the second configuration, an outer dimension at or near the distal portion of the first catheter body is actuated to extend laterally outward by an amount that is sufficient to inhibit backflow of infusate outside of the first catheter body along a tract through tissue through which the first catheter body passes.
